(19) United States
(12) Patent Application Publication
(10) Pub. No.: US 2003/0125269 A1
(43) Pub. Date: Jul. 3, 2003

(54) T-TYPE CALCIUM CHANNEL

(76) Inventor: MING LI, MOBILE, AL (US)

Correspondence Address: SUSAN J BRAMAN ESQ, BRAMAN & ROGALSKYJ LLP, P O BOX 352, CANANDAIGUA, NY, 144240352

(21) Appl. No.: 09/383,894

(22) Filed: Aug. 26, 1999

Related U.S. Application Data
(60) Provisional application No. 60/098,004, filed on Aug. 26, 1998.
(60) Provisional application No. 60/117,399, filed on Jan. 27, 1999.

(51) Int. Cl. A61K 48/00; C07H 21/04; C12P 21/02; C12N 5/06; C07K 14/47

(52) U.S. Cl. 514/44; 536/23.5; 530/350; 435/69.1; 435/320.1; 435/325

(57) ABSTRACT

The present invention is directed to isolated nucleic acid molecules encoding pancreatic T-type calcium channels. Expression vectors and host cells comprising the nucleic acid molecules are also provided, as well as methods for increasing or decreasing the expression of pancreatic T-type calcium channel in host cells. The invention further provides a method of screening a substance for the ability of the substance to modify T-type calcium channel function, and a method for isolating other pancreatic T-type calcium channel molecules. DNA oligomers capable of hybridizing to the nucleic acid molecule encoding the pancreatic T-type calcium channel are provided, which can be used to detect pancreatic T-type calcium channel in a sample. An isolated pancreatic T-type calcium channel protein is also provided. Antibodies specific for the protein, and fragments thereof, are provided, as are compositions comprising the protein and a compatible carrier. The subject invention further provides a method of modifying insulin secretion by pancreatic beta cells, a method of treating type II diabetes in a subject, a method of modifying basal calcium levels in cells, a method of modifying the action potential of L type calcium channels in cells, a method of modifying pancreatic beta cell death, a method of modifying pancreatic beta cell proliferation, and a method of modifying calcium influx through L type calcium channels in cells.

This application claims priority of U.S. Provisional Patent Application No. 60/098,004, filed Aug. 26, 1998, and of U.S. Provisional Patent Application No. 60/117,399, filed Jan. 27, 1999.

The subject matter of this application was made with support from the United States Government under National Institutes of Health Grant No. 5-20174. The U.S. Government may have certain rights in this invention.

### FIELD OF THE INVENTION

The present invention relates generally to calcium channel proteins, and more particularly to pancreatic T-type calcium channel proteins and uses thereof.

### BACKGROUND OF THE INVENTION

Throughout this application various publications are referenced, many in parenthesis. Full citations for each of these publications are provided at the end of the Detailed Description. The disclosures of each of these publications in their entireties are hereby incorporated by reference in this application.

Insulin secretion from pancreatic β-cells is the primary physiological mechanism of blood glucose regulation. A rise in blood glucose concentration stimulates release of insulin from the pancreas, which in turn promotes glucose uptake in peripheral tissues and consequently lowers blood glucose levels, reestablishing euglycemia. Non-insulin dependent diabetes mellitus (NIDDM)(type II diabetes) is associated with an impairment in glucose-induced insulin secretion in pancreatic β-cells (Vague and Moulin, 1982).

Voltage-gated Ca²⁺ channels mediate a rapidly activated inward movement of Ca²⁺ ions that underlies the stimulation of insulin secretion in β-cells (Boyd III 1991). In different tissues, four types of Ca²⁺ channels have been described (L(P/Q), T, N, and E channels). The purified L-type Ca²⁺ channel consists of five subunits: α_{1,} α_{2,} β, γ, δ (Catterall 1991). The primary structure of the α₁ subunit is organized in four homologous domains containing six transmembrane segments (Catterall 1988).

Rat and human pancreatic β-cells are equipped with L-type and T-type Ca²⁺ channels (Hiriart and Matteson, 1988; Davalli et al., 1996). L-type Ca²⁺ channels, activated at high voltages and having large unitary conductance and dihydropyridine-sensitivity, are considered the major pipeline for Ca²⁺ influx into the β-cells (Keahey et al., 1989). In contrast, T-type calcium channels activate at low voltages and have small unitary conductance and dihydropyridine-insensitivity.

The physiological function of T-type Ca²⁺ channels in β-cells insulin-secretion has been demonstrated (Bhattacharjee et al., 1997). These channels facilitate exocytosis by enhancing electrical activity in these cells. L-type and T-type Ca²⁺ channels, under normal conditions, work in concert promoting the rise in [Ca²⁺]ᵢ during glucose-stimulated insulin secretion. In β-cells, over-expressed T-type Ca²⁺ channels may be, at least in part, responsible for the hyper-responsiveness of insulin secretion to non-glucose depolarizing stimuli in GK rat and in rat with NIDDM induced by neonatal injection of streptozotocin (Kato et al., 1994; Kato et al., 1996). However, over-expressed T-type calcium channels over time will ultimately lead to an elevation of basal Ca²⁺ through it's window current properties. Therefore, there is a dual effect of T-type Ca²⁺ channels in β-cells depending upon channel number and membrane potential.

Two isoforms of L-type Ca²⁺ channel al subunits have been identified in β-cells (Seino et al., 1992; Yaney et al., 1992). The rat neuronal T-type calcium channel has recently been cloned (Perez-Reyes et al., 1998). Other subunits of T-type Ca²⁺ channel have yet to be identified.

Given the evidence that T-type calcium channels are associated with type II diabetes, a need exists to further characterize T-type calcium channels.

### SUMMARY OF THE INVENTION

To this end, the subject invention provides an isolated nucleic acid molecule encoding a pancreatic T-type calcium channel. The invention also provides an antisense nucleic acid molecule complementary to at least a portion of the mRNA encoding the pancreatic T-type calcium channel.

The isolated nucleic acid molecules of the invention can be inserted into suitable expression vectors and/or host cells. Expression of the nucleic acid molecules encoding the pancreatic T-type calcium channel results in production of pancreatic T-type calcium channel in a host cell. Expression of the antisense nucleic acid molecules in a host cell results in decreased expression of the pancreatic T-type calcium channel.

The invention further provides a ribozyme having a recognition sequence complementary to a portion of mRNA encoding a pancreatic T-type calcium channel. The ribozyme can be introduced into a cell to also achieve decreased expression of pancreatic T-type calcium channel in the cell.

The invention further provides a method of screening a substance for the ability of the substance to modify T-type calcium channel function, and a method of obtaining DNA encoding a pancreatic T-type calcium channel.

Further provided is an isolated nucleic acid molecule encoding a pancreatic T-type calcium channel, wherein the nucleic acid molecule encodes a first amino acid sequence having at least 90% amino acid identity to a second amino acid sequence. The second amino acid sequence is as shown in SEQ ID NO:2.

The invention further provides a DNA oligomer capable of hybridizing to a nucleic acid molecule encoding a pancreatic T-type calcium channel. The DNA oligomer can be used in a method of detecting presence of a pancreatic T-type calcium channel in a sample, which method is also provided by the subject invention.

The invention also provides an isolated pancreatic T-type calcium channel protein, and antibodies or antibody fragments specific for the pancreatic T-type calcium channel protein. The antibodies and antibody fragments can be used to detect the presence of the pancreatic T-type calcium channel protein in samples. Further provided is an isolated pancreatic T-type calcium channel protein encoded by a first amino acid sequence having at least 90% amino acid identity to a second amino acid sequence, the second amino acid sequence as shown in SEQ ID NO:2.

The subject invention further provides a method of modifying insulin secretion by pancreatic beta cells, the method comprising modifying levels of functional T type calcium channels in the pancreatic beta cells. The invention further provides a method of treating type II diabetes in a subject, the method comprising administering to the subject an amount of a compound effective to modify levels of functional T type calcium channel in the pancreatic beta cells of the subject.

The invention also provides a method of modifying basal calcium levels in cells, a method of modifying the action potential of L type calcium channels in cells, a method of modifying pancreatic beta cell death, a method of modifying pancreatic beta cell proliferation, and a method of modifying calcium influx through L type calcium channels in cells, each of the methods comprising modifying levels of functional T type calcium channels in the cells.

### BRIEF DESCRIPTION OF THE DRAWINGS

These and other features and advantages of this invention will be evident from the following detailed description of preferred embodiments when read in conjunction with the accompanying drawings in which:

FIG. 1A illustrates a comparison of the nucleotide sequence of α₁G-INS (1) and α₁G (2) at the 5′-end regions (aa1-67 of α₁G). The four insertions are indicated with arrow heads. The capital ATG represents the start codon for each cDNA;

FIG. 1B is a schematic illustration representing partial rat genomic nucleotide composition between Domain III and IV. Genomic DNA contained an exon specific to α₁G (shaded circle) and an exon specific to the α₁ subunit of T—type Ca² deduced from INS-1 (shaded rectangle) between 4845 and 5256 of the cDNA sequence. Other exons (open rectangles) are identical between the two cDNAs. The bold letters indicate the nucleotides coding Gly-1667;

FIGS. 2A-2D illustrate expression of α₁G-INS in Xenopus oocytes. FIG. 2A illustrates 40 mM Ca²⁺ currents elicited by depolarizing pulses from −60 to 40 mV. FIG. 2B illustrates time constants of activation and inactivation measured at test potentials between −30 and 30 mV. The time constants of activation were obtained by fitting the increasing portion (activation) of currents with the Hodgkin-Huxley equation where the m value was designated as four (n=6). The time constants of inactivation were obtained by single exponential fitting (n=6). FIG. 2C illustrates voltage-dependent conductance (n=7) and FIG. 2D illustrates steady-state inactivation (n=3) of expressed currents in oocytes. The holding potential for FIGS. 2C and 2D was −80 mV. The currents in FIG. 2D were measured at −10 mV after varying 1000 ms pre-pulse potentials. Peak currents were normalized to the maximum current and then averaged (error bars represent SE);

FIGS. 3A and 3B illustrate accumulative dose response relationships of the inhibitory effects of mibefradil on T- and L-type Ca²⁺ currents. Currents were measured with the whole-cell patch clamp configuration. Data from four experiments were normalized individually and than plotted as mean±standard error. FIG. 3A illustrates curve which was generated by fitting the data using one-to-one binding curve according to the equation 1/(1+[mibefradil]/K_{d}). FIG. 3B is a dose response of L-type Ca²⁺ current obtained when perfusion of solutions containing different concentrations of mibefradil;

FIG. 4 illustrates reversibility of the inhibition of T and L-type currents by NiCl₂ and mibefradil, respectively. Open and solid circles represent the T-type Ca²⁺ current recorded before and after NiCl₂ (2 μl of 30 μM) and mibefradil (2 μl of 10 μM) were administrated, respectively. The open squares represent the L-type Ca²⁺ current recorded before and after mibefradil (2 μl of 10 μM) was administrated with perforated patch clamp configuration. The T-type Ca²⁺ current was measured at −30 mV with a holding potential of −80 mV with whole cell configuration. Arrow indicates the time when the drugs were delivered. n=3 for each group experiments;

FIGS. 5A and 5B illustrate the long-term effect of mibefradil (10 nM) on L- and T-Ca²⁺ currents in the perforated-patch configuration. In FIG. 5A, solid and open circles represent the L-type Ca²⁺ current recorded in the cells with and without administration of mibefradil, respectively. Solid triangles represent T-type Ca²⁺ currents recorded in the cells after administrating mibefradil. Mibefradil were delivered at time zero. n=4 for each group experiments. In FIG. 5B, cells were cultured in medium with or without co-incubating 10 nM mibefradil for 2 hours. The current densities were recorded with perforated patch clamp configuration. n=14 for each group experiments;

FIG. 6A illustrates accumulation of dm-mibefradil in the cells measured with mass spectrometry. The cells were first incubated with mibefradil (20 μM) for the duration indicated on the figure (n=3). The inset (FIG. 6B) shows the primary data of mass spectrometry indicating peaks at 496 and 424, which correspond to mibefradil and dm-mibefradil, respectively;

FIG. 7A illustrates the effect of mibefradil and dm-mibefradil on L-type Ca²⁺ currents from inside cells. n=8, *, p<0.01 to the control;

FIG. 7B illustrates the effect of mibefradil or dm-mibefradil on T-type Ca²⁺ current from inside cells n=4. All data were collected at 5 min after formation of whole cell patch. The pipette solution contained 1 μM of drug;

FIG. 8 illustrates basal [Ca²⁺]ᵢ measured in an INS-1 cell. T-type calcium channel antagonist mibefradil (1 μM) reduced basal [Ca²⁺]ᵢ in a single cell in the bath solution without glucose. The [Ca²⁺]ᵢ was measured with the emission ratio of Fura-2 μM (F380/F340) then calibrated with the standard solution purchased from Molecular Probes Inc. (OR);

FIG. 9A illustrates that intracellular perfusion of a solution containing 272 nM free calcium concentration inhibits the L-type calcium current. Currents were elicited by a step voltage to +10 mV, with holding potential of −80 mV;

FIG. 9B illustrates the effect of perfusing in high calcium concentration on the IV calcium current relationship. Closed circles represent the cell before perfusion, and open circles represent perfusion of 272 nM free calcium;

FIG. 9C illustrates the effect of intracellular perfusion of different calcium concentrations on L-type calcium current over time. Squares represent perfusion from high calcium to low calcium (intracellular solution contained 632 nM then perfused by a solution with 10 μM EGTA), triangles represent perfusion from low calcium to 272 nM calcium, and circles represent low calcium to 632 nM calcium;

FIG. 9D illustrates the effect of high calcium on the T-type calcium channel current. Tail currents were elicited by a voltage step to −30 mV for 10 ms;

FIG. 10 illustrates that reestablishment of basal calcium causes stereotyped calcium influx. A cell was twice perfused with 50 mM KCl with an intervening perfusion of the original bath solution to restore membrane potential;

FIG. 11 illustrates that elevated basal Ca²⁺ causes a defect in the Ca²⁺ transient. A cell was twice perfused with 50 mM KCl with an intervening perfusion of the original bath solution to restore membrane potential. The second perfusion occurred prior to reestablishment of the original basal [Ca²⁺]ᵢ of about 60 nM;

FIG. 12 illustrates a model for glucose-stimulated insulin release;

FIG. 13 illustrates that mibefradil (1 μM) blocks T- and L-type Ca²⁺ current in INS-1 cells. The relative current of T type Ca channel is obtained by measuring their slow deactivated tail current (n=8);

FIG. 14 illustrates that mibefradil and NiCl₂ reversibly block T type Ca²⁺ current in INS-1 cells. Drugs were administered into the recording chamber at 180 seconds from the beginning of recording. N=3;

FIG. 15 illustrates the activation and inactivation curves for INS-1 cells, revealing a “window current”;

FIG. 16 illustrates the effect of NiCl₂, mibefradil, and nifedipine on basal insulin secretion in NIT-1 cells. The glucose concentration is 3 mM in the experiments;

FIG. 17 illustrates that the T type calcium channel antagonist NiCl₂ (30 μM) reduced the frequency of transient spontaneous elevation of [Ca²⁺]ᵢ in a single cell in the bath solution without glucose;

FIG. 18 illustrates the effect of 30 mM NiCl₂ on the [Ca²⁺]ᵢ under non-stimulus conditions. Data was collected from the cells with “high” initial basal [Ca²⁺]ᵢ (about 100 nM). n=13;

FIGS. 19A and 19B illustrate that hyperpolarization induced an increase in number of action potentials and a decrease in onset latencies. N=40;

FIGS. 20A and 20B illustrate the dose-dependent effect of NiCl₂ on insulin secretion. Cells were placed in a medium containing 11.1 mM glucose and a decrease in onset latencies. N=40;

FIG. 21 illustrates “run-up” in whole cell recording;

FIG. 22 illustrates KCl induced Ca²⁺ influx in the INS-1 cells treated with streptozotocin. n=13;

FIGS. 23A-23F illustrate the results of cytokine treatment. LVA Ca²⁺ currents were induced by cytokine treatment (IL-1β, 25 U/ml; IFNγ, 300 U/ml) for 6 h in primary cultured mouse islet cells, but not in α-TC1 cells. An LVA current was elicited by a −40 mV test pulse in an islet cell (FIG. 23A), but the same current was not detected in α-TC1 cells (FIG. 23C). The Ca²⁺ current density-voltage relationships obtained from islet cells (FIG. 23B) and α-TC1 cells (FIG. 23D) with and without cytokine treatment are shown. The open circles represent the current densities of untreated cells (n=10 for islet cells; n=20 for α-TC1 cells), and the filled circles represent the current densities of cells treated by cytokines (n=21 for islet cells; n=21 for α-TC1 cells). The recordings were elicited by voltages ranging from −50 to +20 mV for 100 msec. All experiments were performed at −80 mV. FIG. 23E shows steady state inactivation of LVA tail currents elicited by a 10-msec depolarizing (−10 mV) pulse followed by a 50-msec hyperpolarizing pulse (−100 mV), with a holding potential of −80 mV. FIG. 23F shows that NiCl₂ (10 μM) blocked the cytokine induced LVA Ca²⁺ current elicited at a −30 mV step pulse in an islet cell;

FIGS. 24A and 24B illustrate the effects of cytokines on [Ca²⁺]ᵢ in mouse islet cells and α-TC1 cells. In FIG. 24A, basal [Ca²⁺]ᵢ of primary cultured mouse islet cells was approximately 3-fold higher after cytokine treatment. NiCl₂ (10 μM), but not nifedipine (10 μM) prevented the increase in [Ca²⁺]ᵢ. In FIG. 24B, basal [Ca²⁺] in α-TC1 cells was unaffected by cytokine treatment. Cytokine treatment consisted of IL-1β (25 U/ml) and IFNγ (300 U/ml) for 6 h; and

FIGS. 25A and 25B illustrate the effects of NiCl₂ on cytokine-induced β-TC3 cell death. NiCl₂ (20 μM) significantly reduced cell death induced by cytokines in both a time (FIG. 25A) and dose-dependent (FIG. 25B) manner (n=3). Cytokine treatment consisted of IL-1β (25 U/ml), IFNγ (100 U/ml), and TNFα (100 U/ml) in FIG. 25A and of IL-1β (25 U/ml), TNFα (100 U/ml), and various concentrations of IFNγ as indicated in FIG. 25A. The first dose, 0, represents zero concentration for all three cytokines. The concentration of nifedipine was 10 μM in both FIG. 25A and FIG. 25B.

### DETAILED DESCRIPTION OF THE INVENTION

The term “nucleic acid”, as used herein, refers to either DNA or RNA. “Nucleic acid sequence” or “polynucleotide sequence” refers to a single- or double-stranded polymer of deoxyribonucleotide or ribonucleotide bases read from the 5′ to the 3′ end. It includes both self-replicating plasmids, infectious polymers of DNA or RNA, and nonfunctional DNA or RNA.

“Isolated” nucleic acid refers to nucleic acid which has been separated from an organism in a substantially purified form (i.e. substantially free of other substances originating from that organism), and to synthetic nucleic acid.

By a nucleic acid sequence “homologous to” or “complementary to”, it is meant a nucleic acid that selectively hybridizes, duplexes or binds to DNA sequences encoding the protein (channel) or portions thereof when the DNA sequences encoding the protein are present in a human genomic or cDNA library. A DNA sequence which is similar or complementary to a target sequence can include sequences which are shorter or longer than the target sequence so long as they meet the functional test set forth.

Typically, the hybridization is done in a Southern blot protocol using a 0.2×SSC, 0.1% SDS, 65° C. wash. The term “SSC” refers to a citrate-saline solution of 0.15M sodium chloride and 20 mM sodium citrate. Solutions are often expressed as multiples or fractions of this concentration. For example, 6×SSC refers to a solution having a sodium chloride and sodium citrate concentration of 6 times this amount or 0.9 M sodium chloride and 120 mM sodium citrate. 0.2×SSC refers to a solution 0.2 times the SSC concentration or 0.03M sodium chloride and 4 mM sodium citrate.

The phrase “nucleic acid molecule encoding” refers to a nucleic acid molecule which directs the expression of a specific protein or peptide. The nucleic acid sequences include both the DNA strand sequence that is transcribed into RNA and the RNA sequence that is translated into protein or peptide. The nucleic acid molecule includes both the full length nucleic acid sequences as well as non-full length sequences derived from the full length protein. It being further understood that the sequence includes the degenerate codons of the native sequence or sequences which may be introduced to provide codon preference in a specific host cell.

The term “located upstream” as used herein refers to linkage of a promoter upstream from a nucleic acid (DNA) sequence such that the promoter mediates transcription of the nucleic acid (DNA) sequence.

The term “vector”, refers to viral expression systems, autonomous self-replicating circular DNA (plasmids), and includes both expression and nonexpression plasmids. Where a recombinant microorganism or cell is described as hosting an “expression vector,” this includes both extrachromosomal circular DNA and DNA that has been incorporated into the host chromosome(s). Where a vector is being maintained by a host cell, the vector may either be stably replicated by the cells during mitosis as an autonomous structure, or the vector may be incorporated within the host's genome.

The term “plasmid” refers to an autonomous circular DNA molecule capable of replication in a cell, and includes both the expression and nonexpression types. Where a recombinant microorganism or cell is described as hosting an “expression plasmid”, this includes latent viral DNA integrated into the host chromosome(s). Where a plasmid is being maintained by a host cell, the plasmid is either being stably replicated by the cell during mitosis as an autonomous structure, or the plasmid is incorporated within the host's genome.

The phrase “heterologous protein” or “recombinantly produced heterologous protein” refers to a peptide or protein of interest produced using cells that do not have an endogenous copy of DNA able to express the peptide or protein of interest. The cells produce the peptide or protein because they have been genetically altered by the introduction of the appropriate nucleic acid sequences. The recombinant peptide or protein will not be found in association with peptides or proteins and other subcellular components normally associated with the cells producing the peptide or protein.

The following terms are used to describe the sequence relationships between two or more nucleic acid molecules or polynucleotides, or between two or more amino acid sequences of peptides or proteins: “reference sequence”, “comparison window”, “sequence identity”, “sequence homology”, “percentage of sequence identity”, “percentage of sequence homology”, “substantial identity”, and “substantial homology”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence.

Optimal alignment of sequences for aligning a comparison window may be conducted, for example, by the local homology algorithm of Smith and Waterman (1981), by the homology alignment algorithm of Needleman and Wunsch (1970), by the search for similarity method of Pearson and Lipman (1988), or by computerized implementations of these algorithms (GAP, BESTFIT, FASTA, and TFASTA in the Wisconsin Genetics Software Package Release 7.0, Genetics Computer Group, 575 Science Dr., Madison, Wis.).

As applied to nucleic acid molecules or polynucleotides, the terms “substantial identity” or “substantial sequence identity” mean that two nucleic acid sequences, when optimally aligned (see above), share at least 90 percent sequence identity, preferably at least 95 percent sequence identity, more preferably at least 96, 97, 98 or 99 percent sequence identity.

“Percentage nucleotide (or nucleic acid) identity” or “percentage nucleotide (or nucleic acid) sequence identity” refers to a comparison of the nucleotides of two nucleic acid molecules which, when optimally aligned, have approximately the designated percentage of the same nucleotides. For example, “95% nucleotide identity” refers to a comparison of the nucleotides of two nucleic acid molecules which when optimally aligned have 95% nucleotide identity. Preferably, nucleotide positions which are not identical differ by redundant nucleotide substitutions (the nucleotide substitution does not change the amino acid encoded by the particular codon).

As further applied to nucleic acid molecules or polynucleotides, the terms “substantial homology” or “substantial sequence homology” mean that two nucleic acid sequences, when optimally aligned (see above), share at least 90 percent sequence homology, preferably at least 95 percent sequence homology, more preferably at least 96, 97, 98 or 99 percent sequence homology.

“Percentage nucleotide (or nucleic acid) homology” or “percentage nucleotide (or nucleic acid) sequence homology” refers to a comparison of the nucleotides of two nucleic acid molecules which, when optimally aligned, have approximately the designated percentage of the same nucleotides or nucleotides which are not identical but differ by redundant nucleotide substitutions (the nucleotide substitution does not change the amino acid encoded by the particular codon). For example, “95% nucleotide homology” refers to a comparison of the nucleotides of two nucleic acid molecules which when optimally aligned have 95% nucleotide homology.

As applied to polypeptides, the terms “substantial identity” or “substantial sequence identity” mean that two peptide sequences, when optimally aligned, such as by the programs GAP or BESTFIT using default gap, share at least 90 percent sequence identity, preferably at least 95 percent sequence identity, more preferably at least 96, 97, 98 or 99 percent sequence identity.

“Percentage amino acid identity” or “percentage amino acid sequence identity” refers to a comparison of the amino acids of two polypeptides which, when optimally aligned, have approximately the designated percentage of the same amino acids. For example, “95% amino acid identity” refers to a comparison of the amino acids of two polypeptides which when optimally aligned have 95% amino acid identity. Preferably, residue positions which are not identical differ by conservative amino acid substitutions. For example, the substitution of amino acids having similar chemical properties such as charge or polarity are not likely to affect the properties of a protein. Examples include glutamine for asparagine or glutamic acid for aspartic acid.

As further applied to polypeptides, the terms “substantial homology” or “substantial sequence homology” mean that two peptide sequences, when optimally aligned, such as by the programs GAP or BESTFIT using default gap, share at least 90 percent sequence homology, preferably at least 95 percent sequence homology, more preferably at least 96, 97, 98 or 99 percent sequence homology.

“Percentage amino acid homology” or “percentage amino acid sequence homology” refers to a comparison of the amino acids of two polypeptides which, when optimally aligned, have approximately the designated percentage of the same amino acids or conservatively substituted amino acids. For example, “95% amino acid homology” refers to a comparison of the amino acids of two polypeptides which when optimally aligned have 95% amino acid homology. As used herein, homology refers to identical amino acids or residue positions which are not identical but differ only by conservative amino acid substitutions. For example, the substitution of amino acids having similar chemical properties such as charge or polarity are not likely to affect the properties of a protein. Examples include glutamine for asparagine or glutamic acid for aspartic acid.

The phrase “substantially purified” or “isolated” when referring to a protein (or peptide), means a chemical composition which is essentially free of other cellular components. It is preferably in a homogeneous state although it can be in either a dry or aqueous solution. Purity and homogeneity are typically determined using analytical chemistry techniques such as polyacrylamide gel electrophoresis or high performance liquid chromatography. A protein (or peptide) which is the predominant species present in a preparation is substantially purified. Generally, a substantially purified or isolated protein (or peptide) will comprise more than 80% of all macromolecular species present in the preparation. Preferably, the protein (or peptide) is purified to represent greater than 90% of all macromolecular species present. More preferably the protein (or peptide) is purified to greater than 95%, and most preferably the protein (or peptide) is purified to essential homogeneity, wherein other macromolecular species are not detected by conventional techniques. A “substantially purified” or “isolated” protein (or peptide) can be separated from an organism, synthetically or chemically produced, or recombinantly produced.

“Biological sample” or “sample” as used herein refers to any sample obtained from a living organism or from an organism that has died. Examples of biological samples include body fluids and tissue specimens.

High stringent hybridization conditions are selected at about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may significantly affect the stringency of hybridization, including, among others, base composition and size of the complementary strands, the presence of organic solvents, i.e. salt or formamide concentration, and the extent of base mismatching, the combination of parameters is more important than the absolute measure of any one. High stringency may be attained, for example, by overnight hybridization at about 68° C. in a 6×SSC solution, washing at room temperature with 6×SSC solution, followed by washing at about 68° C. in a 6×SSC solution then in a 0.6×SSX solution.

Hybridization with moderate stringency may be attained, for example, by: 1) filter pre-hybridizing and hybridizing with a solution of 3×sodium chloride, sodium citrate (SSC), 50% formamide, 0.1M Tris buffer at pH 7.5, 5× Denhardt's solution; 2) pre-hybridization at 37° C. for 4 hours; 3) hybridization at 37° C. with amount of labeled probe equal to 3,000,000 cpm total for 16 hours; 4) wash in 2×SSC and 0.1% SDS solution; 5) wash 4×for 1 minute each at room temperature and 4×at 60° C. for 30 minutes each; and 6) dry and expose to film.

The phrase “selectively hybridizing to” refers to a nucleic acid molecule that hybridizes, duplexes or binds only to a particular target DNA or RNA sequence when the target sequences are present in a preparation of total cellular DNA or RNA. By selectively hybridizing it is meant that a nucleic acid molecule binds to a given target in a manner that is detectable in a different manner from non-target sequence under moderate, or more preferably under high, stringency conditions of hybridization. “Complementary” or “target” nucleic acid sequences refer to those nucleic acid sequences which selectively hybridize to a nucleic acid molecule. Proper annealing conditions depend, for example, upon a nucleic acid molecule's length, base composition, and the number of mismatches and their position on the molecule, and must often be determined empirically. For discussions of nucleic acid molecule (probe) design and annealing conditions, see, for example, Sambrook et al. 1989.

It will be readily understood by those skilled in the art and it is intended here, that when reference is made to particular sequence listings, such reference includes sequences which substantially correspond to its complementary sequence and those described including allowances for minor sequencing errors, single base changes, deletions, substitutions and the like, such that any such sequence variation corresponds to the nucleic acid sequence of the signal peptide or other peptide/protein to which the relevant sequence listing relates.

The DNA molecules of the subject invention also include DNA molecules coding for protein analogs, fragments or derivatives of the protein which differ from naturally-occurring forms (the naturally-occurring protein) in terms of the identity or location of one or more amino acid residues (deletion analogs containing less than all of the residues specified for the protein, substitution analogs wherein one or more residues specified are replaced by other residues, and addition analogs wherein one or more amino acid residues is added to a terminal or medial portion of the protein) and which share the signal property of the naturally-occurring form. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors.

As used herein, a “peptide” refers to an amino acid sequence of three to one hundred amino acids, and therefore an isolated peptide that comprises an amino acid sequence is not intended to cover amino acid sequences of greater than 100 amino acids. Preferably, the peptides that can be identified and used in accordance with the subject invention (whether they be mimotope or anti-mimotope peptides) are less than 50 amino acids in length, and more preferably the peptides are five to 20 amino acids in length or 20-40 amino acids in length.

The peptides can contain any naturally-occurring or non-naturally-occurring amino acids, including the D-form of the amino acids, amino acid derivatives and amino acid mimics, so long as the desired function and activity of the peptide is maintained. The choice of including an (L)- or a (D)-amino acid in the peptides depends, in part, on the desired characteristics of the peptide. For example, the incorporation of one or more (D)-amino acids can confer increased stability on the peptide and can allow a peptide to remain active in the body for an extended period of time. The incorporation of one or more (D)-amino acids can also increase or decrease the pharmacological activity of the peptide.

The peptides may also be cyclized, since cyclization may provide the peptides with superior properties over their linear counterparts.

As used herein, the terms “amino acid mimic” and “mimetic” mean an amino acid analog or non-amino acid moiety that has the same or similar functional characteristic of a given amino acid. For instance, an amino acid mimic of a hydrophobic amino acid is one which is non-polar and retains hydrophobicity, generally by way of containing an aliphatic chemical group. By way of further example, an arginine mimic can be an analog of arginine which contains a side chain having a positive charge at physiological pH, as is characteristic of the guanidinium side chain reactive group of arginine.

In addition, modifications to the peptide backbone and peptide bonds thereof are also encompassed within the scope of amino acid mimic or mimetic. Such modifications can be made to the amino acid, derivative thereof, non-amino acid moiety or the peptide either before or after the amino acid, derivative thereof or non-amino acid moiety is incorporated into the peptide. What is critical is that such modifications mimic the peptide backbone and bonds which make up the same and have substantially the same spacial arrangement and distance as is typical for traditional peptide bonds and backbones. An example of one such modification is the reduction of the carbonyl(s) of the amide peptide backbone to an amine. A number of reagents are available and well known for the reduction of amides to amines such as those disclosed in Wann et al., JOC, 46:257 (1981) and Raucher et al., Tetrahedron. Lett., 21:14061 (1980). An amino acid mimic is, therefor, an organic molecule that retains the similar amino acid pharmacophore groups as is present in the corresponding amino acid and which exhibits substantially the same spatial arrangement between functional groups.

The substitution of amino acids by non-naturally occurring amino acids and amino acid mimics as described above can enhance the overall activity or properties of an individual peptide based on the modifications to the backbone or side chain functionalities. For example, these types of alterations to the specifically described amino acid substituents and exemplified peptides can enhance the peptide's stability to enzymatic breakdown and increase biological activity. Modifications to the peptide backbone similarly can add stability and enhance activity.

One skilled in the art, using the above sequences or formulae, can easily synthesize the peptides. Standard procedures for preparing synthetic peptides are well known in the art. The novel peptides can be synthesized using: the solid phase peptide synthesis (SPPS) method of Merrifield (J. Am. Chem. Soc., 85:2149 (1964)) or modifications of SPPS; or, the peptides can be synthesized using standard solution methods well known in the art (see, for example, Bodanzsky, M., Principles of Peptide Synthesis, 2nd revised ed., Springer-Verlag (1988 and 1993)). Alternatively, simultaneous multiple peptide synthesis (SMPS) techniques well known in the art can be used. Peptides prepared by the method of Merrifield can be synthesized using an automated peptide synthesizer such as the Applied Biosystems 431A-01 Peptide Synthesizer (Mountain View, Calif.) or using the manual peptide synthesis technique described by Houghten, Proc. Natl. Acad. Sci., USA 82:5131 (1985).

With these definitions in mind, the subject invention provides an isolated nucleic acid molecule encoding a pancreatic T-type calcium channel. The nucleic acid molecule can be deoxyribonucleic acid (DNA) or ribonucleic acid (RNA, including messenger RNA or mRNA), genomic or recombinant, biologically isolated or synthetic.

The DNA molecule can be a cDNA molecule, which is a DNA copy of a messenger RNA (mRNA) encoding the channel.

An example of such a pancreatic T-type calcium channel is the rat pancreatic T-type calcium channel encoded by the nucleotide sequence as shown in SEQ ID NO:1. The amino acid sequence encoded by this nucleotide sequence is shown in SEQ ID NO:2.

The invention also provides an antisense nucleic acid molecule that is complementary to at least a portion of the mRNA encoding the pancreatic T-type calcium channel. Antisense nucleic acid molecules can be RNA or single-stranded DNA, and can be complementary to the entire mRNA molecule encoding the channel (i.e. of the same nucleotide length as the entire molecule). It may be desirable, however, to work with a shorter molecule. In this instance, the antisense molecule can be complementary to a portion of the entire mRNA molecule encoding the channel. These shorter antisense molecules are capable of hybridizing to the mRNA encoding the entire molecule, and preferably consist of about twenty to about one hundred nucleotides. These antisense molecules can be used to reduce levels of pancreatic T-type calcium channel, by introducing into cells an RNA or single-stranded DNA molecule that is complementary to at least a portion of the mRNA of the channel (i.e. by introducing an antisense molecule). The antisense molecule can base-pair with the mRNA of the channel, preventing translation of the mRNA into protein. Thus, an antisense molecule to the channel can prevent translation of mRNA encoding the channel into a functional channel protein. It may be desirable to place the antisense molecule downstream and under the control or the insulin promoter, so that the antisense will prevent translation of mRNA encoding the T type calcium channel only in islet cells of the pancreas (not affecting brain or heart T type calcium channels). It should also be apparent that 100% prevention of T type calcium channel is not desirable, since a minimal basal Ca²⁺ level is required to be maintained by the T type calcium channel.

More particularly, an antisense molecule complementary to at least a portion of mRNA encoding a pancreatic T-type calcium channel can be used to decrease expression of a functional channel. A cell with a first level of expression of a functional pancreatic T-type calcium channel is selected, and then the antisense molecule is introduced into the cell. The antisense molecule blocks expression of functional pancreatic T-type calcium channel, resulting in a second level of expression of a functional pancreatic T-type calcium channel in the cell. The second level is less than the initial first level.

Antisense molecules can be introduced into cells by any suitable means. In one embodiment, the antisense RNA molecule is injected directly into the cellular cytoplasm, where the RNA interferes with translation. A vector may also be used for introduction of the antisense molecule into a cell. Such vectors include various plasmid and viral vectors. For a general discussion of antisense molecules and their use, see Han et al. 1991 and Rossi 1995.

The invention further provides a special category of antisense RNA molecules, known as ribozymes, having recognition sequences complementary to specific regions of the mRNA encoding the pancreatic T-type calcium channel. Ribozymes not only complex with target sequences via complementary antisense sequences but also catalyze the hydrolysis, or cleavage, of the template mRNA molecule. Examples, which are not intended to be limiting, of suitable regions of the mRNA template to be targeted by ribozymes are any of the homologous regions identified by comparing the various T-type calcium channels, and particularly pancreatic β-cell T-type channels.

Expression of a ribozyme in a cell can inhibit gene expression (such as the expression of a pancreatic T-type calcium channel). More particularly, a ribozyme having a recognition sequence complementary to a region of a mRNA encoding a pancreatic T-type calcium channel can be used to decrease expression of pancreatic T-type calcium channel. A cell with a first level of expression of pancreatic T-type calcium channel is selected, and then the ribozyme is introduced into the cell. The ribozyme in the cell decreases expression of pancreatic T-type calcium channel in the cell, because mRNA encoding the pancreatic T-type calcium channel is cleaved and cannot be translated.

Ribozymes can be introduced into cells by any suitable means. In one embodiment, the ribozyme is injected directly into the cellular cytoplasm, where the ribozyme cleaves the mRNA and thereby interferes with translation. A vector may be used for introduction of the ribozyme into a cell. Such vectors include various plasmid and viral vectors (note that the DNA encoding the ribozyme does not need to be “incorporated” into the genome of the host cell; it could be expressed in a host cell infected by a viral vector, with the vector expressing the ribozyme, for instance). For a general discussion of ribozymes and their use, see Sarver et al. 1990, Chrisey et al. 1991, Rossi et al. 1992, and Christoffersen et al. 1995.

The nucleic acid molecules of the subject invention can be expressed in suitable host cells using conventional techniques. Any suitable host and/or vector system can be used to express the pancreatic T-type calcium channel. For in vitro expression, Xenopus oocytes are preferred. For in vivo expression, the most suitable host cell is a pancreatic β-cell.

Techniques for introducing the nucleic acid molecules into the host cells may involve the use of expression vectors which comprise the nucleic acid molecules. These expression vectors (such as plasmids and viruses; viruses including bacteriophage) can then be used to introduce the nucleic acid molecules into suitable host cells. For example, DNA encoding the pancreatic T-type calcium channel can be injected into the nucleus of a host cell or transformed into the host cell using a suitable vector, or mRNA encoding the pancreatic T-type calcium channel can be injected directly into the host cell, in order to obtain expression of pancreatic T-type calcium channel in the host cell.

Various methods are known in the art for introducing nucleic acid molecules into host cells. One method is microinjection, in which DNA is injected directly into the nucleus of cells through fine glass needles (or RNA is injected directly into the cytoplasm of cells). Alternatively, DNA can be incubated with an inert carbohydrate polymer (dextran) to which a positively charged chemical group (DEAE, for diethylaminoethyl) has been coupled. The DNA sticks to the DEAE-dextran via its negatively charged phosphate groups. These large DNA-containing particles stick in turn to the surfaces of cells, which are thought to take them in by a process known as endocytosis. Some of the DNA evades destruction in the cytoplasm of the cell and escapes to the nucleus, where it can be transcribed into RNA like any other gene in the cell. In another method, cells efficiently take in DNA in the form of a precipitate with calcium phosphate. In electroporation, cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures. DNA can also be incorporated into artificial lipid vesicles, liposomes, which fuse with the cell membrane, delivering their contents directly into the cytoplasm. In an even more direct approach, DNA is absorbed to the surface of tungsten microprojectiles and fired into cells with a device resembling a shotgun.

Several of these methods, microinjection, electroporation, and liposome fusion, have been adapted to introduce proteins into cells. For review, see Mannino and Gould-Fogerite 1988, Shigekawa and Dower 1988, Capecchi 1980, and Klein et al. 1987.

Further methods for introducing nucleic acid molecules into cells involve the use of viral vectors. One such virus widely used for protein production is an insect virus, baculovirus. For a review of baculovirus vectors, see Miller (1989). Various viral vectors have also been used to transform mammalian cells, such as bacteriophage, vaccinia virus, adenovirus, and retrovirus.

As indicated, some of these methods of transforming a cell require the use of an intermediate plasmid vector. U.S. Pat. No. 4,237,224 to Cohen and Boyer describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture. The DNA sequences are cloned into the plasmid vector using standard cloning procedures known in the art, as described by Sambrook et al. (1989).

Host cells into which the nucleic acid encoding the pancreatic T-type calcium channel has been introduced can be used to produce (i.e. to functionally express) the pancreatic T-type calcium channel. The function of the encoded pancreatic T-type calcium channel can be assayed according to methods known in the art (Wang et al. 1996).

Having identified the nucleic acid molecules encoding pancreatic T-type calcium channels and methods for expressing the pancreatic T-type calcium channels encoded thereby, the invention further provides a method of screening a substance (for example, a compound or inhibitor) for the ability of the substance to modify T-type calcium channel function. The method comprises introducing a nucleic acid molecule encoding the pancreatic T-type calcium channel into a host cell, and expressing the pancreatic T-type calcium channel encoded by the molecule in the host cell. The cell is then exposed to a substance and evaluated to determine if the substance modifies the function of the T-type calcium channel. From this evaluation, substances effective in altering the function of the T-type calcium channel can be found. Such agents may be, for example, calcium channel inhibitors, agonists, or antagonists (for example, mibefradil and mibefradil analogues, amiloride, NiCl₂, antisense molecules, and second messengers).

The evaluation of the cell to determine if the substance modifies the function of the T-type calcium channel can be by any means known in the art. The evaluation can comprise the direct monitoring of expression of T-type calcium channel in the host cell, or the evaluation can be indirect and comprise the monitoring of calcium transport by the channel (such as by the methods disclosed by Wang et al. 1996).

The nucleic acid molecules of the subject invention can be used either as probes or for the design of primers to obtain DNA encoding other pancreatic T-type calcium channels by either cloning and colony/plaque hybridization or amplification using the polymerase chain reaction (PCR).

Specific probes derived from SEQ ID NO:1 can be employed to identify colonies or plaques containing cloned DNA encoding a member of the pancreatic T-type calcium channel family using known methods (see Sambrook et al. 1989). One skilled in the art will recognize that by employing such probes under high stringency conditions (for example, hybridization at 42° C. with 5×SSPC and 50% formamide, washing at 50-65° C. with 0.5×SSPC), sequences having regions which are greater than 90% homologous or identical to the probe can be obtained. Sequences with lower percent homology or identity to the probe, which also encode pancreatic T-type calcium channels, can be obtained by lowering the stringency of hybridization and washing (e.g., by reducing the hybridization and wash temperatures or reducing the amount of formamide employed).

More particularly, in one embodiment, the method comprises selection of a DNA molecule encoding a pancreatic T-type calcium channel, or a fragment thereof, the DNA molecule having a nucleotide sequence as shown in SEQ ID NO:1, and designing an oligonucleotide probe for pancreatic T-type calcium channel based on SEQ ID NO:1. A genomic or cDNA library of an organism is then probed with the oligonucleotide probe, and clones are obtained from the library that are recognized by the oligonucleotide probe so as to obtain DNA encoding another pancreatic T-type calcium channel.

Specific primers derived from SEQ ID NO:1 can be used in PCR to amplify a DNA sequence encoding a member of the pancreatic T-type calcium channel family using known methods (see Innis et al. 1990). One skilled in the art will recognize that by employing such primers under high stringency conditions (for example, annealing at 50-60° C., depending on the length and specific nucleotide content of the primers employed), sequences having regions greater than 75% homologous or identical to the primers will be amplified.

More particularly, in a further embodiment the method comprises selection of a DNA molecule encoding pancreatic T-type calcium channel, or a fragment thereof, the DNA molecule having a nucleotide sequence as shown in SEQ ID NO:1, designing degenerate oligonucleotide primers based on regions of SEQ ID NO:1, and employing such primers in the polymerase chain reaction using as a template a DNA sample to be screened for the presence of pancreatic T-type calcium channel-encoding sequences. The resulting PCR products can be isolated and sequenced to identify DNA fragments that encode polypeptide sequences corresponding to the targeted region of pancreatic T-type calcium channel.

Various modifications of the nucleic acid and amino acid sequences disclosed herein are covered by the subject invention. These varied sequences still encode a functional pancreatic T-type calcium channel. The invention thus further provides an isolated nucleic acid molecule encoding a pancreatic T-type calcium channel, the nucleic acid molecule encoding a first amino acid sequence having at least 90% amino acid identity to a second amino acid sequence, the second amino acid sequence as shown in SEQ ID NO:2. In further embodiments, the first amino acid sequence has at least 95%, 96%, 97%, 98%, or 99% amino acid identity to SEQ ID NO:2.

The invention further provides an isolated DNA oligomer capable of hybridizing to the nucleic acid molecule encoding pancreatic T-type calcium channel according to the subject invention. Such oligomers can be used as probes in a method of detecting the presence of pancreatic T-type calcium channel in a sample. More particularly, a sample can be contacted with the DNA oligomer and the DNA oligomer will hybridize to any pancreatic T-type calcium channel present in the sample, forming a complex therewith. The complex can then be detected, thereby detecting presence of pancreatic T-type calcium channel in the sample.

The complex can be detected using methods known in the art. Preferably, the DNA oligomer is labeled with a detectable marker so that detection of the marker after the DNA oligomer hybridizes to any pancreatic T-type calcium channel in the sample (wherein non-hybridized DNA oligomer has been washed away) is detection of the complex. Detection of the complex indicates the presence of pancreatic T-type calcium channel in the sample. As will be readily apparent to those skilled in the art, such a method could also be used quantitatively to assess the amount of pancreatic T-type calcium channel in a sample.

For detection, the oligomers can be labeled with, for example, a radioactive isotope, biotin, an element opaque to X-rays, or a paramagnetic ion. Radioactive isotopes are commonly used and are well known to those skilled in the art. Representative examples include indium-111, technetium-99m, and iodine-123. Biotin is a standard label which would allow detection of the biotin labeled oligomer with avidin. Paramagnetic ions are also commonly used and include, for example, chelated metal ions of chromium (III), manganese (II), and iron (III). When using such labels, the labeled DNA oligomer can be imaged using methods known to those skilled in the art. Such imaging methods include, but are not limited to, X-ray, CAT scan, PET scan, NMRI, and fluoroscopy. Other suitable labels include enzymatic labels (horseradish peroxidase, alkaline phosphatase, etc.) and fluorescent labels (such as FITC or rhodamine, etc.).

The invention further provides an isolated pancreatic T-type calcium channel protein. The protein is preferably encoded by a nucleotide sequence as shown in SEQ ID NO:1. The protein preferably has an amino acid sequence as shown in SEQ ID NO:2. Further provided is an isolated pancreatic T-type calcium channel protein encoded by a first amino acid sequence having at least 90% amino acid identity to a second amino acid sequence, the second amino acid sequence as shown in SEQ ID NO:2. In further embodiments, the first amino acid sequence has at least 95%, 96%, 97%, 98%, or 99% amino acid identity to SEQ ID NO:2.

The pancreatic T-type calcium channel molecule of the subject invention can include a leader sequence for targeting of the pancreatic T-type calcium channel protein to the desired part of a cell.

It should be readily apparent to those skilled in the art that a met residue may need to be added to the amino terminal of the amino acid sequence of the mature pancreatic T-type calcium channel protein (i.e., added to SEQ ID NO:2) or an ATG added to the 5′ end of the nucleotide sequence (i.e., added to SEQ ID NO:1), in order to express the channel in a host cell. The met version of the mature channel is thus specifically intended to be covered by reference to SEQ ID NO:1 or SEQ ID NO:2.

The invention further provides an antibody or fragment thereof specific for the pancreatic T-type calcium channel of the subject invention. Antibodies of the subject invention include polyclonal antibodies and monoclonal antibodies capable of binding to the pancreatic T-type calcium channel, as well as fragments of these antibodies, and humanized forms. Humanized forms of the antibodies of the subject invention may be generated using one of the procedures known in the art such as chimerization. Fragments of the antibodies of the present invention include, but are not limited to, the Fab, the F(ab′)₂, and the Fc fragments.

The invention also provides hybridomas which are capable of producing the above-described antibodies. A hybridoma is an immortalized cell line which is capable of secreting a specific monoclonal antibody.

In general, techniques for preparing polyclonal and monoclonal antibodies as well as hybridomas capable of producing the desired antibody are well known in the art (see Campbell 1984 and St. Groth et al. 1980). Any animal (mouse, rabbit, etc.) which is known to produce antibodies can be immunized with the antigenic pancreatic T-type calcium channel (or an antigenic fragment thereof). Methods for immunization are well known in the art. Such methods include subcutaneous or intraperitoneal injection of the protein. One skilled in the art will recognize that the amount of the protein used for immunization will vary based on the animal which is immunized, the antigenicity of the protein, and the site of injection.

The protein which is used as an immunogen may be modified or administered in an adjuvant in order to increase the protein's antigenicity. Methods of increasing the antigenicity of a protein are well known in the art and include, but are not limited to, coupling the antigen with a heterologous protein (such as a globulin or beta-galactosidase) or through the inclusion of an adjuvant during immunization.

For monoclonal antibodies, spleen cells from the immunized animals are removed, fused with myeloma cells, such as SP2/O-Ag 15 myeloma cells, and allowed to become monoclonal antibody producing hybridoma cells.

Any one of a number of methods well known in the art can be used to identify the hybridoma cell which produces an antibody with the desired characteristics. These include screening the hybridomas with an ELISA assay, western blot analysis, or radioimmunoassay (Lutz et al. 1988).

Hybridomas secreting the desired antibodies are cloned and the class and subclass are determined using procedures known in the art (Campbell 1984).

For polyclonal antibodies, antibody containing antisera is isolated from the immunized animal and is screened for the presence of antibodies with the desired specificity using one of the above-described procedures.

The present invention further provides the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.), fluorescent labels (such as FITC or rhodamine, etc.), paramagnetic atoms, etc. Procedures for accomplishing such labeling are well known in the art, for example see Sternberger et al. 1970, Bayer et al. 1979, Engval et al. 1972, and Goding 1976.

The labeled antibodies or fragments thereof of the present invention can be used for in vitro, in vivo, and in situ assays to identify cells or tissues which express pancreatic T-type calcium channel, to identify samples containing pancreatic T-type calcium channel, or to detect the presence of pancreatic T-type calcium channel in a sample. More particularly, the antibodies or fragments thereof can thus be used to detect the presence of pancreatic T-type calcium channel in a sample, by contacting the sample with the antibody or fragment thereof. The antibody or fragment thereof binds to any pancreatic T-type calcium channel present in the sample, forming a complex therewith. The complex can then be detected, thereby detecting the presence of pancreatic T-type calcium channel in the sample. As will be readily apparent to those skilled in the art, such a method could also be used quantitatively to assess the amount of pancreatic T-type calcium channel in a sample. As should also be readily apparent, such an antibody can also be used to decrease levels of functional T type calcium channels, by blocking the channel. Such antibodies can therefore be used in the methods of the subject invention to modify levels of functional T type calcium channels in pancreatic beta cells.

Further provided is a composition comprising the pancreatic T-type calcium channel protein and a compatible carrier.

In the methods of the invention, tissues or cells are contacted with or exposed to the composition of the subject invention or a compound. In the context of this invention, to “contact” tissues or cells with or to “expose” tissues or cells to a composition or compound means to add the composition or compound, usually in a liquid carrier, to a cell suspension or tissue sample, either in vitro or ex vivo, or to administer the composition or compound to cells or tissues within an animal (including humans).

For therapeutics, methods of modifying insulin secretion by pancreatic beta cells, methods of treating type II diabetes, methods of modifying basal calcium levels in cells, methods of modifying the action potential of L type calcium channels in cells, methods of modifying pancreatic beta cell death, methods of modifying pancreatic beta cell proliferation, and methods of modifying calcium influx through L type calcium channels in cells, each of the methods comprising modifying levels of functional T type calcium channels in the cells, are provided. The formulation of therapeutic compositions and their subsequent administration is believed to be within the skill in the art. In general, for therapeutics, a patient suspected of needing such therapy is given a composition in accordance with the invention, commonly in a pharmaceutically acceptable carrier, in amounts and for periods which will vary depending upon the nature of the particular disease, its severity and the patient's overall condition. The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip or infusion, subcutaneous, intraperitoneal or intramuscular injection, pulmonary administration, e.g., by inhalation or insufflation, or intrathecal or intraventricular administration.

Formulations for topical administration may include transdermal patches, ointments, lotions, creams, gels, drops, suppositories, sprays, liquids and powders.

Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral, intrathecal or intraventricular administration may include sterile aqueous solutions which may also contain buffers, diluents and other suitable additives.

In addition to such pharmaceutical carriers, cationic lipids may be included in the formulation to facilitate uptake. One such composition shown to facilitate uptake is LIPOFECTIN (BRL, Bethesda Md.).

Dosing is dependent on severity and responsiveness of the condition to be treated, with course of treatment lasting from several days to several months or until a cure is effected or a diminution of disease state is achieved. Optimal dosing schedules can be calculated from measurements of drug accumulation in the body. Persons of ordinary skill can easily determine optimum dosages, dosing methodologies and repetition rates. Optimum dosages may vary depending on the relative potency of individual compositions, and can generally be calculated based on IC₅₀'s or EC₅₀'s in in vitro and in vivo animal studies. For example, given the molecular weight of compound (derived from oligonucleotide sequence and/or chemical structure) and an effective dose such as an IC₅₀, for example (derived experimentally), a dose in mg/kg is routinely calculated.

The methods of the subject invention are based on the discovery that regulation of T type calcium channels directly modifies basal calcium levels in cells, which in turn regulates L type calcium channel activity, which in turn regulates insulin secretion and cell death, which in turn treats type II diabetes. The methods of the subject invention are further based on the discovery that regulation of T type calcium channels directly affects basal and glucose-induced insulin secretion.

T type calcium channels belong to the family of low voltage activated calcium channels. Modifying (increasing or decreasing) “levels” of functional T type calcium channels refers to modifying expression of the T type calcium channel gene, modifying activity of the T type calcium channel such as by inhibiting the function of the channel, and/or modifying the formation of active membrane-spanning T type calcium channels. As used herein, “functional” refers to the synthesis and any necessary post-translational processing of a calcium channel molecule in a cell so that the channel is inserted properly in the cell membrane and is capable of conducting calcium ions in accordance with a low voltage activated channel.

The invention thus provides a method of modifying insulin secretion by pancreatic beta cells, the method comprising modifying levels of T type calcium channels in the pancreatic beta cells.

Levels of T type calcium channels in the pancreatic beta cells can be modified by various methods, at the gene and protein and “functional calcium channel” levels. In one embodiment, the levels are modified by modifying T type calcium channel gene expression of the T type calcium channel in the cells. This can be accomplished by exposing the cells to a compound which modifies T type calcium channel gene expression of the calcium channel. The compound could be, for example, an antisense oligonucleotide targeted to the T type calcium channel gene. In a similar embodiment, the compound which modifies T type calcium channel gene expression of the T type calcium channel could be a ribozyme.

Other methods for modifying T type calcium channel gene expression could also involve site-directed mutagenesis of the T type calcium channel gene to prevent expression of the T type calcium channel, or various gene therapy techniques.

Levels, in particular activity, of T type calcium channels in the cell can also be modified by exposing the cells to an inhibitor of the T type calcium channel. Such inhibitors include, for example, mibefradil, mibefradil analogs, amiloride, NiCl₂, and second messengers which regulate activity of the T type calcium channels. Other inhibitors of the T type calcium channel could also readily be identified by screening methods (including the method described above). In addition to chemical inhibitors, peptide inhibitors could also be identified with screening methods (for example, using phage display libraries and other peptide screening methods).

“Mibefradil analogs”, as used herein are meant to include compounds having the formula:

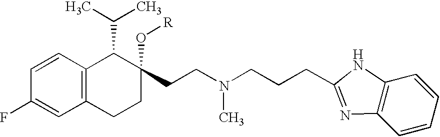

wherein R is hydrogen, alkyl, or a moiety having the formula C(O)R′, where R′ is alkyl or aryl. In the above formulae, alkyl is meant to include linear alkyls, particularly C1-C12 linear alkyls (e.g., methyl, ethyl, n—propyl, n-pentyl, n-hexyl, n-heptyl, n-octyl, n-nonyl, and the like), branched alkyls, particularly C1-C12 branched alkyls (e.g., isobutyl, isopentyl, neopentyl, hex-2-yl, hex-3-yl, hept-2-yl, hept-3-yl, and the like), and cycloalkyls, particularly C1-C8 cycloalkyls (e.g., cyclopentyl, cyclohexyl, cycloheptyl, 4-methylcyclohexyl, and the like). These alkyl groups can be substituted or unsubstituted. When substituted, suitable substituents include, for example, aryl groups, halogen atoms, hydroxy groups, alkoxy groups, carboxylic acid groups, amine groups, and the like, as well as combinations of these substituents. Mibefradil analogs which are particularly well suited to blocking (inhibiting) the activity of T-type calcium channels but not blocking the activity of L-type calcium channels are those having the formula:

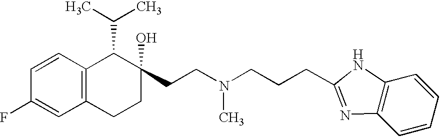

and those having the formula:

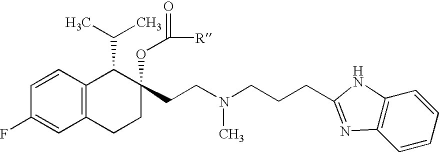

in which R″ is an unsubstituted alkyl group or a substituted alkyl group which does not contain an alkoxy substituent. “Mibefradil analogs” are also meant to include compounds having the above formulae which are substituted at other positions in the structure, for example, on the benzimidazole phenyl moiety, at a benzimidazole nitrogen, at other positions of the tetrahydronaphthyl ring, etc. Also included within the meaning of “mibefradil analogs” are compounds having the above formulae in which the F is replaced with another substituent, such as another halogen. Also included within the meaning of “mibefradil analogs” are compounds having the above formulae in which the amine methyl group or the isopropyl group or both are replaced with other substituents, such as other alkyl moieties. Additionally, “mibefradil analogs” are meant to include those compounds which are generically described and/or specifically disclosed in U.S. Pat. No. 4,808,605, which is hereby incorporated by reference. Further, “mibefradil analogs” are meant to include pharmaceutically acceptable salts of the derivatives described above. Illustrative pharmaceutically acceptable salts are salts formed with hydrochloric acid, hydrobromic acid, nitric acid, sulphuric acid, phosphoric acid, citric acid, formic acid, maleic acid, acetic acid, succinic acid, tartaric acid, methanesulphonic acid, p-toluenesulphonic, and the like.

Mibefradil analogs can be made by following the general procedures described in, for example, U.S. Pat. Nos. 4,808,605, 5,910,606, 5,892,055, 5,811,557, 5,811,556, and 5,808,088, each of which is hereby incorporated by reference.

Levels of T type calcium channels in the cell can also be modified by exposing the cells to a compound which interferes with membrane T type calcium channel formation.

Levels of functional T type calcium channel could also be modified by use of molecules which bind to transcription regulators of the T type calcium channel gene (such as the promoter region of the gene).

The invention further provides a method of treating type II diabetes in a subject (human or animal), the method comprising administering to the subject an amount of a compound effective to modify levels of T type calcium channels in the pancreatic beta cells of the subject. As above, the compound may modify levels of T type calcium channels by modifying T type calcium channel gene expression of the calcium channel, or by inhibiting the T type calcium channel, or by interfering with membrane T type calcium channel formation.

In the context of this invention “modulation” or “modifying” means either inhibition or stimulation. This modulation can be measured in ways which are routine in the art, for example by Northern blot assay of mRNA expression, Western blot assay of protein expression, or calcium channel activity assay.

The compounds and/or inhibitors used in the methods of the subject invention encompass any pharmaceutically acceptable salts, esters, or salts of such esters, or any other compound/inhibitor which, upon administration to an animal including a human, is capable of providing (directly or indirectly) the biologically active metabolite or residue thereof. Accordingly, for example, the disclosure is also drawn to prodrugs and pharmaceutically acceptable salts of the compounds and/or inhibitors used in the subject invention, pharmaceutically acceptable salts of such prodrugs, and other bioequivalents.

In regard to prodrugs, the compounds and/or inhibitors for use in the invention may additionally or alternatively be prepared to be delivered in a prodrug form. The term prodrug indicates a therapeutic agent that is prepared in an inactive form that is converted to an active form (i.e., drug) within the body or cells thereof by the action of endogenous enzymes or other chemicals and/or conditions.

In regard to pharmaceutically acceptable salts, the term pharmaceutically acceptable salts refers to physiologically and pharmaceutically acceptable salts of the compounds and/or inhibitors used in the subject invention: i.e., salts that retain the desired biological activity of the parent compound and do not impart undesired toxicological effects thereto.

Drugs, such as peptide drugs, which inhibit the T type calcium channel or which interfere with functional T type calcium channel formation can be identified by other methods also. For example, a monoclonal antibody can be prepared which specifically hybridizes to the T type calcium channel, thereby interfering with activity and/or channel formation. Once a monoclonal antibody which specifically hydridizes to the T type calcium channel is identified, the monoclonal (which is itself a compound or inhibitor which can be used in the subject invention) can be used to identify peptides capable of mimicking the inhibitory activity of the monoclonal antibody. One such method utilizes the development of epitope libraries and biopanning of bacteriophage libraries. Briefly, attempts to define the binding sites for various monoclonal antibodies have led to the development of epitope libraries. Parmley and Smith developed a bacteriophage expression vector that could display foreign epitopes on its surface (Parmley, S. F. & Smith, G. P., Gene 73:305-318 (1988)). This vector could be used to construct large collections of bacteriophage which could include virtually all possible sequences of a short (e.g. six-amino-acid) peptide. They also developed biopanning, which is a method for affinity-purifying phage displaying foreign epitopes using a specific antibody (see Parmley, S. F. & Smith, G. P., Gene 73:305-318 (1988); Cwirla, S. E., et al., Proc Natl Acad Sci USA 87:6378-6382 (1990); Scott, J. K. & Smith, G. P., Science 249:386-390 (1990); Christian, R. B., et al., J Mol Biol 227:711-718 (1992); Smith, G. P. & Scott, J. K., Methods in Enzymology 217:228-257 (1993)).

After the development of epitope libraries, Smith et al. then suggested that it should be possible to use the bacteriophage expression vector and biopanning technique of Parmley and Smith to identify epitopes from all possible sequences of a given length. This led to the idea of identifying peptide ligands for antibodies by biopanning epitope libraries, which could then be used in vaccine design, epitope mapping, the identification of genes, and many other applications (Parmley, S. F. & Smith, G. P., Gene 73:305-318 (1988); Scott, J. K., Trends in Biochem Sci 17:241-245 (1992)).

Using epitope libraries and biopanning, researchers searching for epitope sequences found instead peptide sequences which mimicked the epitope, i.e., sequences which did not identify a continuous linear native sequence or necessarily occur at all within a natural protein sequence. These mimicking peptides are called mimotopes. In this manner, mimotopes of various binding sites/proteins have been found.

The sequences of these mimotopes, by definition, do not identify a continuous linear native sequence or necessarily occur in any way in a naturally-occurring molecule, i.e. a naturally occurring protein. The sequences of the mimotopes merely form a peptide which functionally mimics a binding site on a naturally-occurring protein.

Many of these mimotopes are short peptides. The availability of short peptides which can be readily synthesized in large amounts and which can mimic naturally-occurring sequences (i.e. binding sites) offers great potential application.

Using this technique, mimotopes to a monoclonal antibody that recognizes T type calcium channels can be identified. The sequences of these mimotopes represent short peptides which can then be used in various ways, for example as peptide drugs that bind to T type calcium channels and decrease the activity of T type calcium channels. Once the sequence of the mimotope is determined, the peptide drugs can be chemically synthesized.

Materials and Methods

Cell Culture—INS-1 cells were cultured in RPMI 1640 medium containing 10% FBS, 25 U/ml penicillin, 25 mg/ml streptomycin and 50 μM mercaptoethanol in an atmosphere of 5% CO₂ in air, at 37° C. for 2-5 days before recording.

Islet cell preparation—Pancreases of Sprague-Dawley rats (Charles River Laboratory, Wilmington, Mass.) were removed after intrapancreatic perfusion with 2 ml of Hanks' solution (Gibco BRL, Grand Island, N.Y.) containing collagenase (4 mg/ml, Boehringer Mannheim, Indianapolis, Ind.), DNase I (10 μg/ml, Sigma, St. Louis, Mo.), CaCl₂ (1.28 mM) and bovine serum albumin (1L mg/ml, Gibco BRL). The pancreatic tissue was incubated at 37° C. for 20 min and then washed five times with enzyme-free Hanks' solution. Islets were picked up and treated with 0.1% pancreatin (Sigma) for five minutes at 37° C. Single cells were obtained by triturating the islets with plastic pipette tips and then they were transferred into 35 mm culture dishes. Cells were cultured in RPMI 1640 medium (Gibco BRL) containing 5 mM glucose, 10% FBS and P/S at 37° C., 5% CO₂ for 2-5 days before experiments.

Isolation of RNA—Total RNA was isolated from cultured INS-1 cells and from various freshly excised rat tissues by the guanidinium isothiocyanate/phenol procedure (Chomczynsk and Sacchi 1987). Poly-A RNA was isolated from total RNA by two successive passes over an oligo (dT)-cellulose spin column (Ambion, Austin, Tex.).

Cloning of cDNA Encoding al Subunit of T-type Ca²⁺ channel in INS-1—First strand cDNA was prepared using 2 μg of INS-1 cell mRNA and M-MLV reverse transcriptase (Gibco BRL) with the poly-dT primers. The first 433 bp DNA fragment of the channel was deduced with PCR using the degenerate primers (forward)(SEQ ID NO:6) 5′-TNGC(A/C/T)ATGGAG(C/A)GNCC(C/T)-3′ and (backward)(SEQ ID NO:7) 5′-CTT(C/G/T)CCCTTGAA(G/C)A(G/A)CTG)-3′ based on conserved voltage-dependent Ca²⁺ channel α₁ subunit sequences in domain III. Using the Marathon™ cDNA Amplification Kit (Clontech, Palo Alto, Calif.), the 31—and 51—rapid amplifications of cDNA end-PCR (RACE-PCR) were performed to obtain the entire gene of the α₁ subunit of the channel. For the 5′-RACE-PCR, the forward primer was an adapter primer, the backward primer was (SEQ ID NO:8) 5′-CCGCTGTCGGAGACCATGGAGACC-3′; for the 3′-RACE, the forward primer was (SEQ ID NO:9) 5′-AGCGGCCCAAAATTGACCCCCACAG-3′ and the backward primer was poly-dT. The RT-PCR products were subcloned into pT-Adv Vector (Clontech) and dideoxynucleotide sequencing assay was performed with a dsDNA Cycle Sequencing System (Gibco BRL).

Tissue distribution—The gene expression of T-type Ca²⁺ channels deduced from β-cells was examined in rat brain, heart, kidney, and liver using an RT-PCR assay. The primers used for the RT-PCR were (SEQ ID NO:10) 5′-GAAGATGCGAGTGGACAG-3′ (forward) and (SEQ ID NO:1l) 5′-CTGTGGCGATGGTCACTG-3′ (backward). The PCR products were detected by agarose gel electrophoresis on a 1% gel.

Genome walking—The genome walker library (Clontech) was used as a template in nested PCR reactions with gene-specific primers (GSP) and the adapter primers (AP) provided with the kit. The first PCR reaction was carried out in 5 tubes, each having a total volume of 50 μl: 5yl 10×PCR reaction buffer, 1 μl dNTP (10 mM each), 2.2 μl Mg(OAc)₂ (25 mM), 1 μl AP1 (10 μM), 1 μl GSP1, 1 μl Advantage Genomic Polymerase Mix (50×), and 37.8 μl water. The following two-step cycle parameters were used: (Step 1) 7 cycles of denaturing at 94° C. for 25 sec., annealing and extension at 72° C. for 4 min. (Step 2) 32 cycles of denaturing at 94° C. for 25 sec., annealing and extension at 67° C. for 4 min. After the second step cycle, the samples were held at 67° C. for 4 min. The second PCR reaction was carried out under the reaction condition similar to the first PCR reaction except using AP2, GSP2. In addition, the templates used were 1 μl of 1:50 dilution of each primary PCR reaction. The two step cycles were similar to the first PCR reaction except 5 cycles at the first step and 22 cycles at the second step.

Oocyte electrophysiology—cRNA transcripts were synthesized from BssH II linearized pT-Adv cDNA templates using T7 RNA polymerase (Ambion). Defolliculated Xenopus laevis were injected with 25 ng pT-Adv cRNA. Three to five days after injection, two-electrode voltage-clamp recording was performed using a Warner OC-725C amplifier (Warner Instrument Corp., Hamden, Conn.). Data were acquired and analyzed with Pulse/PulseFit software (HEKA, Lambrecht/Pfalz, Germany). The bath solution contained the following: 40 mM Ca(OH)₂, 50 mM NaOH, 2 mM TEA-Cl, 1 mM KOH, 0.1 mM EDTA and 5 mM HEPES, adjusted to pH 7.4 with methanesulphonate. Boltzmann fits were calculated using Prism (GraphPad). Results are presented as mean±s.d. unless otherwise stated.

β-cells Electrophysiological recording—The whole-cell recordings were carried out by the standard “giga-seal” patch clamp technique (Hamill et al.). The whole-cell recording pipettes were made of hemocapillaries (Warner), pulled by a two-stage puller (PC-10, Narishige International, New York, N.Y.), and heat polished with a microforge (MF200-1, World Precision Instruments, Sarasota, Fla.) before use. Pipette resistance was in the range of 2-5 MΩ in the internal solution. The recordings were performed at room temperature (22-25° C.). Currents were recorded using an EPC-9 patch-clamp amplifier (HEKA) and filtered at 2.9 kHz. Data were acquired with Pulse/PulseFit software (HEKA). Voltage-dependent currents were corrected for linear leak and residual capacitance by using an on-line P/n subtraction paradigm.

Drugs—Mibefradil ((1S,2S)-2-[2-[[3-(2-Benzimidazolyl)propyl]methyl-amino]ethyl]-6-fluoro-1,2,3,4-tetrahydro-1-isopropyl-2-napthyl methoxy-acetate dihydrochloride) was kindly provided by Dr. J.-P. Clozel (Hoffmann LaRoche, Basel, Switzerland), and can be synthesized according to the methods disclosed in U.S. Pat. Nos. 5,892,055, 5,811,557, 5,811,556, and 5,808,088. U.S. Pat. No. 4,808,605 describes mibefradil compounds suitable for use in the subject invention.

The free alcohol Des-methoxyacetyl mibefradil (1S,2S)-2-[2-[[3-(2-Benzimidazolyl)propyl]methylamino]ethyl]-6-fluoro-1,2,3,4-tetrahydro-1-isopropyl-2-napthyl hydroxy hydrochloride) was prepared by alkaline hydrolysis: 14.2 mg mibefradil hydrochloride was dissolved in 4 ml methanol +1 ml 10 N aqueous sodium hydroxide mixture (5 mM was the final concentration of mibefradil). The solution was warmed in a boiling water bath for 10 min. The reaction was followed by mass spectrometry. Upon completion of the hydrolysis, as determined from the mass spectra, the solution was neutralized with 5 M aqueous hydrochloric acid. The slight loss of methanol that occurred by evaporation during the reaction was corrected by adding water to keep the total volume of 5 ml.

Solutions—The extracellular solution used in whole-cell Ca²⁺ current recording contained (in mM): 10 CaCl₂₁ 110 tetraethylammonium-Cl (TEA-Cl), 10 CsCl, 10 N-2-hydroxyethylpiperazine-N′-2-ethanesulfonic acid (HEPES), 40 sucrose, 0.5 3,4-diaminopyridine, pH 7.3. The intracellular solution contained (in mM): 130 N-methyl-D-glucamine, 20 EGTA (free acid), 5 bis (2-aminophenoxy) ethane-N,N,N′,N′-tetraacetate (BAPTA), 10 HEPES, 6 MgCl₂, 4 Ca(OH)₂/pH was adjusted to 7.4 with methanesulfonate. 2 mM Mg-ATP was included in the pipette solution to minimize rundown of L-type Ca²⁺ currents. For Perforated-patch recording, the extracellular solution contained (in mM): 26 Sucrose, 30 TEA-Cl, 10 HEPES, 5 KCl, 2 CaCl₂, MgCl₂, pH 7.3. The pipette solution contained (in mM): 65 CsOH, 65 CsMS, 20 sucrose, 10 HEPES, 10 MgCl₂, 1 Ca(OH)₂, pH 7.4.

Mass Spectrometric Analysis—A VG 70-250 SEQ instrument (VG Analytical, Manchaster, UK) was used with fast atom bombardment (FAB) ionization mode to obtain mass spectra of the mibefradil and dm-mibefradil. Cultured INS-1 cells were treated with 20 μM mibefradil for various lengths of time under each experimental condition. The cell pellets were collected after washing three times with PBS and resuspended in 0.5 ml media for mass spectrometric analysis. For a 50 μl cell sample, 20 μl internal standard solution (40 μM verapamil, MW: 454) and 5 μl glycerol was added, and 4 μl of this mixture was used for FAB-MS. Several positive ion spectra were recorded in the mass range m/z 750-100 at a mass resolution of 1000, and a scan speed of 2 second/decade. For mibefradil, m/z 496 was the dominant ion (M+H)⁺ accompanied with a less intense sodiated molecular ion m/z 518. The concentrations of the mibefradil and hydrolyzed mibefradil were obtained by comparing the intensities of m/z 496 and 424 were to the intensity of m/z 455. For calibration, a standard solution of 50 μM drug was subjected to mass spectrometric analysis.

Separation of cytosolic and membrane components—After washing out mibefradil from the bath solution, the cells were collected and the membranes were broken down by vortexing the cells in a solution containing 5% acetic acid/CH₃CN. The mixture was then spun and the supernatant collected and defined as non-membrane associated components. Pellets were re-suspended in 5× volume of NaOH (10 N):methanol (1:7) solution at 37° C. for 5 min. The mixture was neutralized with 0.5 M HCl and spun down. The remaining pellet and the supernatant were collected separately.

Statistics—All data is presented as mean±s.d. and the student's t-test was used to calculate p values where given.

### EXAMPLE I

### Identification and Cloning of a Pancreatic T-type Calcium Channel

The subject invention provides a cDNA encoding a T-type Ca²⁺ channel α₁ subunit derived from the rat insulin secreting cell line, INS-1, which has been identified and sequenced. The sequence of the cDNA indicates a protein composed of 2288 amino acids (SEQ ID NO:2), sharing 96.3% identity to the neuronal T-type Ca²⁺ channel α₁ subunit (α₁G). The transmembrane domains of the protein are highly conserved but the isoform contains three distinct regions as well as 10 single amino acid substitutions in other regions. Sequencing rat genomic DNA revealed that this is an alternative splice isoform of α₁G. Using specific primers and reverse transcription polymerase chain reaction (RT-PCR) it was demonstrated that both splice variants are expressed in rat islets. The isoform deduced from INS-1 was also expressed in brain, neonatal heart and kidney. Functional expression of this α₁G isoform in Xenopus oocytes generated low-voltage activated Ca²⁺ currents. These results provide the molecular biological basis for studies of function of T-type Ca²⁺ channels in β-cells where these channels play critical roles in diabetes.

The cloning and tissue distribution of an isoform of the T-type Ca²⁺ channel (α₁G-INS) derived from the rat insulin-secreting cell line, INS-1 (Asfari et al. 1992), is described further below.

Based on the conserved amino acid sequence comprising the six transmembrane segments in repeat III of the previously cloned α₁-subunit (Stea et al. 1995), degenerate primers were designed to deduce the cDNA sequence of voltage-dependent Ca²⁺ channel from INS-1 which expresses a high level of T-type Ca²⁺ current (Bhattacharjee et al. 1997). A 433 base pair (bp) DNA fragment was obtained. The rapid amplification of cDNA ends (RACE) strategy was then used to obtain the entire sequence of the channel. The full length cDNA (SEQ ID NO:1) encodes a protein containing 2288 amino acids (SEQ ID NO:2).

The T-type Ca²⁺ channel gene deduced from β-cells shares 96.3% amino acid identity with α₁G, the neuronal isoform of T-type Ca²⁺ channel (Perez-Reyes et al. 1998). The four intramolecular homologous transmembrane domains of β-cells T-type Ca²⁺ channel α₁ subunit are identical (except glycine 1667) to α₁G, with each repeat containing six putative membrane-spanning regions (S1-S6) and a pore-forming region (P-loop). The other highly conserved region is located at the intracellular loop between repeat I and II, where a section of histidine-rich chain is present in the β-cell derived T-type Ca²⁺ channel gene as well as in neuronal and cardiac T-type Ca²⁺ channel genes. This structure in the loop_{I-II} has not been observed in the protein sequences of known high voltage activated Ca²⁺ channels.

In addition to the single amino acids that differ from α₁G, the T-type Ca²⁺ channel gene derived from β-cells contains three unique regions that differ from the amino acid sequence of α₁G. These regions are located at the N-terminal amino acids (aa1-34 of SEQ ID NO:2), intracellular loop I_{II-III} (aa971-994 of SEQ ID NO:2) and intracellular loop L_{III-IV} (aa1570-1588 of SEQ ID NO:2).

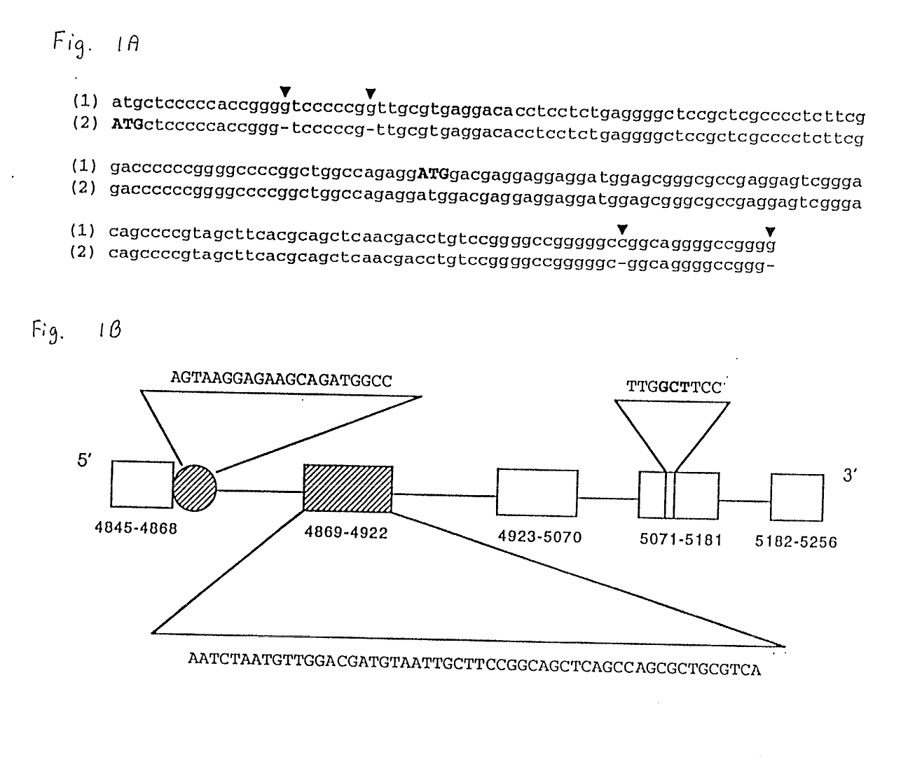

Although the amino acid sequence of the deduced channel is entirely different from the α₁G in the N-terminal region (aa1-34 of SEQ ID NO:2), the nucleotide sequences at this region are almost identical except for 4 single nucleotide insertions which are shown in FIG. 1A. These four single nucleotide insertions determine a different start codon as well as those of the amino acid sequences.

To resolve the relationship between the T-type Ca²⁺ channel isoform deduced from INS-1 and α₁G, a section of Sprague-Dawley rat genomic DNA sequence containing the introns and exons between 4845 and 5256 was identified. As shown in FIG. 1B, an exon was found that encodes the α₁G fragment SKEKQMA (SEQ ID NO:5) as well as an exon that encodes fragment 4869-4922 of the INS-1 variant. This region also contains 8.5 kilobases (kb) of intron sequence. Thus, the T-type Ca²⁺ channel α₁ subunit cloned from INS-1 and α₁G are alternative splice isoforms of the same gene.

The genomic DNA sequence was also used to examine the two nucleotide discrepancy between the α₁G cDNA and the isoform cloned from INS-1. The data show that the genomic nucleotide sequence encoding amino acid 1667 is GGC (glycine), which is the same as the cDNA of α₁ subunit cloned from INS-1 and the corresponding residue in α₁H, but is different from α₁G (GCG, alanine). Also of note, there are nine additional single amino acid substitutions in the isoform deduced from INS-1 as compared to the α₁G. Six correspond to the amino acids found in the analogous position of α₁H: cysteine 1088, glycine 1667, alanine 1700, aspartic acid 1735, threonine 1812, and leucine 1813.

In regard to tissue distribution of T-type Ca²⁺ channels deduced from β-cells and from neurons, expression of the β-cells T-type Ca²⁺ channel was found in rat brain, heart and kidney, but was absent from liver. Both α₁G and the splice form were detected in rat islets and INS-1 cell preparations using RT-PCR. No α₁H was detected.

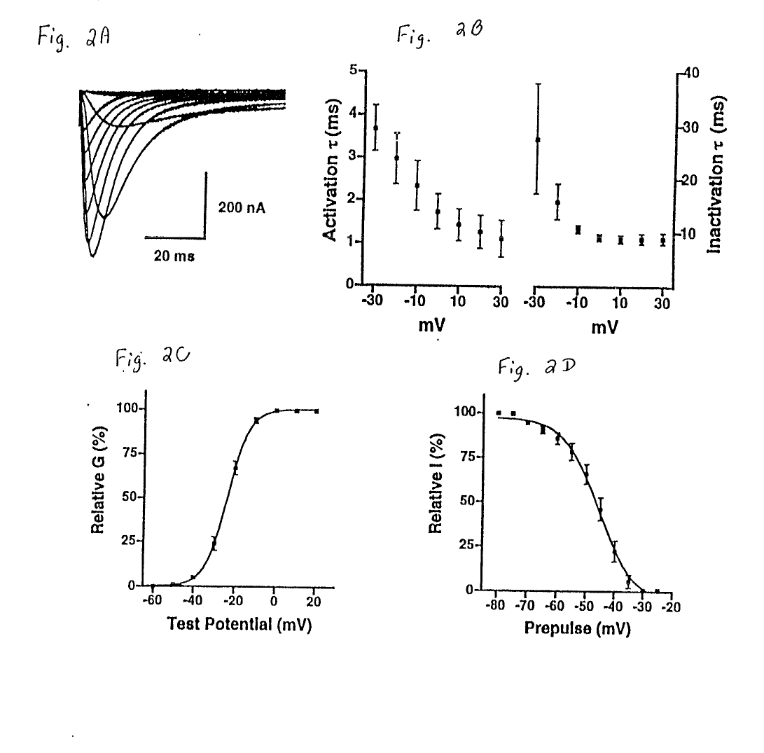

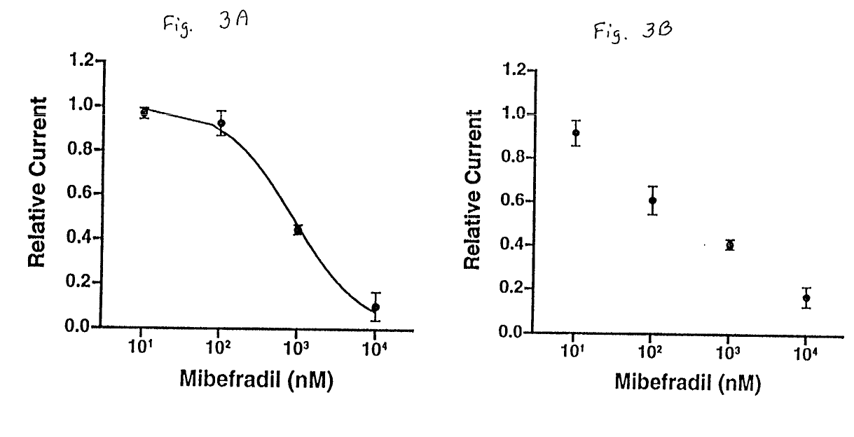

Functional expression of the T-type Ca²⁺ channels deduced from β-cells has been conducted in Xenopus oocytes using a double-electrode voltage-clamp method. In a solution containing 40 mM Ca²⁺ , a family of current traces representing T-type Ca²⁺ current characteristics were obtained (FIG. 2A). The current slowly activated at −40 mV and peaked at −10 mV. The analysis of time constants of activation and inactivation are shown in FIG. 2B. The voltage-dependent activation (FIG. 2C) and steady-state inactivation (FIG. 2D) were fitted with Boltzmann equation. The calculated V_{1/2}'s were −23.8 mV and

45.6 mV for activation and inactivation, respectively; and k's were 5.3 and −6.0 for activation and inactivation, respectively.

The nucleotide cDNA (SEQ ID NO:1) and amino acid (SEQ ID NO:2) sequences of rat pancreatic T-type calcium channel were determined. SEQ ID NO:3 is the nucleotide sequence beyond the coding region, while SEQ ID NO:4 includes SEQ ID NO:2.

### EXAMPLE II

### Characterization of the T type Calcium Channel in Relation to Diabetes

Glucose stimulated insulin release is Ca²⁺ dependent process, involving closure of the ATP-sensitive potassium channels, depolarization and opening of the voltage-dependent Ca²⁺ channels. At glucose concentrations below 3 mM, which do not elicit insulin secretion, β-cells are electrically silent with a resting membrane potential of about −70 mV. Raising external glucose produces a slow depolarization, the extend dependent upon the glucose concentration. At glucose levels which elicit insulin release (>7 mM) depolarization is sufficient to reach the threshold potential (−50 mV) at which electrical activity is initiated.

A simple model for glucose-stimulated insulin secretion is summarized in FIG. 12. The resting membrane potential of β-cells is principally determined by the activity of the K-ATP channel. When plasma glucose rises, its uptake and rate of metabolism by β-cells are stimulated. As a consequence, the intracellular ATP (or ATP:ADP ratio) increases which leads to the closure of K-ATP channels and membrane depolarization. This results in the activation of voltage dependent Ca²⁺ channels (T-type and L-type) and the initiation of electrical activity. The increased calcium influx leads to a rise in [Ca²⁺]ᵢ and consequently insulin secretion.

Rat and human pancreatic β-cells are equipped with L-type and T-type Ca² channels. The physiological function of T-type Ca²⁺ channels in β-cells insulin-secretion has been demonstrated. These channels facilitate exocytosis by enhancing electrical activity in these cells. L-type and T-type Ca²⁺ channels, under normal conditions, work in concert promoting the rise in [Ca²⁺]ᵢ during glucose-stimulated insulin secretion. In β-cells, over-expressed T-type Ca²⁺ channels are, at least in part, responsible for the hyper-responsiveness of insulin secretion to non-glucose depolarizing stimuli in GK rat, and in rat with NIDDM induced by neonatal injection of streptozotocin. However, over-expressed T-type calcium channels over time will ultimately lead to an elevation of basal Ca²⁺ through its window current properties. Therefore, there is a dual effect of T-type Ca²⁺ channels in β-cells depending upon channel number and membrane potential.

Pharmacologically antagonizing T-type calcium channels is an appropriate treatment protocol for alleviating both insulin resistance and enhancement of insulin secretion in NIDDM patients.

NIDDM pathogenesis is complex and the disease progression occurs in phases. An enhanced β-cell responsiveness provokes and initiates the disease process. It is unclear as to what the actual enhanced activity is and what the triggering mechanisms are for this first phase. It may be an increased secretory response or an increase in β-cells mass. However, there is clearly an enhancement of β-cells activity detected by both basal and postprandial elevated insulin levels denoted as hyperinsulinemia. Consequently, a resulting insulin resistance occurs, phase II, particularly in insulin responsive tissues (muscle, liver, kidney, fat) that function to reduce glucose levels in the blood. A decrease in insulin sensitivity will account for an increase in blood glucose, causing the β-cells to secrete even more insulin to compensate and because of this vicious cycle, full blown NIDDM, marked by an inevitable defect in insulin release, hyperglycemia and insulin resistance, will characterize the final stage of the disease process.

Each phase of the disease may be characterized by an alteration in [Ca²⁺]ᵢ, and each phase can be treated by a T-type calcium channel antagonist. The electrical β-cells is equipped with two types of voltage-dependent calcium channels, L-type and T-type calcium channels. L-type calcium channels, activated at high voltages, having large unitary conductance, and dihydropyridine-sensitive, are considered the major pipeline for calcium influx into the β-cells (especially at high voltage depolarization). T-type calcium channels, activated at low voltages, with small unitary conductance, and dihydropyridine-insensitive, are important for maintaining basal [Ca²⁺]ᵢ (FIG. 8), as well as enhancing electrical activity during cell depolarization. T-type calcium channels normally facilitate insulin secretion in β-cells by enhancing cell electrical activity. This modulatory function of T-type calcium channels in insulin secretion is significant during phase I prior to onset of diabetes. Antagonizing these T-type calcium channels will decrease β-cells hyper-responsiveness and consequent hyperinsulinemia arresting the pathogenic pathways that lead to NIDDM.

If hyperinsulinemia and associated insulin resistance has already occurred, a T-type calcium channel blocker is still the appropriate treatment protocol. The insulin responsive tissues, those that are primarily responsible for taking up glucose for re-establishing euglycemia, have elevated basal [Ca²⁺]ᵢ during hyperinsulinemic conditions. Indeed, it is the elevated basal [Ca²⁺]ᵢ that precipitates the decrease in insulin sensitivity of these tissues and it is now known that most of these insulin responsive tissues express T-type calcium channels. A T-type calcium channel blocker will reduce the basal [Ca²⁺]ᵢ and alleviate the decreased insulin sensitivity.

Once NIDDM has manifested, it is characterized by altered glucose metabolism, a result of abnormal glucose stimulus-secretion responsiveness of β-cells. β-cell desensitization to glucose is the principal secretory defect of NIDDM. L-type and T-type calcium channels, under normal conditions, work in concert promoting the rise in [Ca²⁺]ᵢ during glucose-stimulated insulin secretion. In NIDDM, this partnership is broken and the necessary rise in [Ca²⁺]ᵢ for insulin secretion is compromised.

The data herein indicates that L-type calcium channels are finely regulated by basal calcium levels (FIGS. 9A-9D). A very small rise in basal calcium will substantially decrease the L-type calcium current and severely reduce the depolarization-induced rise in [Ca²⁺]ᵢ (FIGS. 10 and 11). The data herein also suggests that T-type calcium channels are a primary regulator of resting basal [Ca²⁺]ᵢ in β-cells. Furthermore, the negative feedback regulation of T-type calcium channels by elevated [Ca²⁺]ᵢ is absent (FIGS. 9A-9D). It is under circumstances of enhanced T-type calcium current activity as seen in the GK rat model of NIDDM and in the neonate streptozotocin-induced diabetes model, that basal [Ca²⁺]ᵢ is elevated, and a defect in the glucose-stimulated insulin secretion is observed. Simply reducing the basal calcium influx by pharmacological intervention, in situations of enhanced T-type calcium channel expression, may reduce basal [Ca²⁺]ᵢ in β-cells (FIG. 8) and alleviate the [Ca²⁺]ᵢ-induced inhibition of L-type calcium channels.

There is a clear link between [Ca²⁺]ᵢ and diabetes. A primary abnormality in [Ca²⁺]ᵢ handling by cells is the defect initiating parallel impairments in insulin secretion and insulin action, as well as initiating diabetic complications. Consequent metabolic derangements may further aggravate alterations in [Ca²⁺]ᵢ homeostasis, creating a relentless cycle leading to progressive deterioration in the overall health of the diabetic patient. Pharmacological agents that regulate [Ca²⁺]ᵢ homeostasis are thus appropriate therapeutic measures. The use of T-type calcium channel blockers will thus effectively treat and perhaps cure diabetes mellitus.

### EXAMPLE III

### Pharmacology of Mibefradil Action

It has been shown that mibefradil has a potent inhibitory effect on T-type Ca²⁺ current in vascular smooth muscle cells. The data herein demonstrates that, in convention whole cell patch clamp configuration, mibefradil also blocks T-type Ca²⁺ current in pancreatic -cells. Mibefradil (1 μM) had been administered in the recording chamber at time zero (FIG. 13), the control (no drug) showed “run down”. This figure shows that T-type Ca²⁺ current is more sensitive to mibefradil than the L-type Ca²⁺ current in pancreatic β-cells.

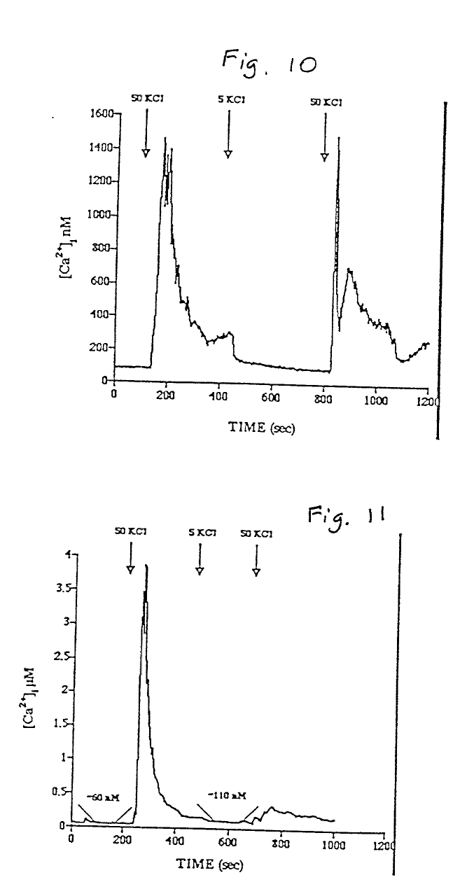

The blockade of T-type Ca²⁺ channels in β-cells with mibefradil is reversible. FIG. 14 demonstrates the reversibility of blockade of T-type Ca²⁺ currents by mibefradil. In these experiments, a very little volume of mibefradil or NiCl₂ was delivered near the recording cell. The drug then diffused away from the cell. The final concentration in the chamber was 1 nM. This experiment shows the inhibitory effect of mibefradil on T-type Ca²⁺ current in pancreatic β-cells results from reversible interaction between the drug and the channel protein.

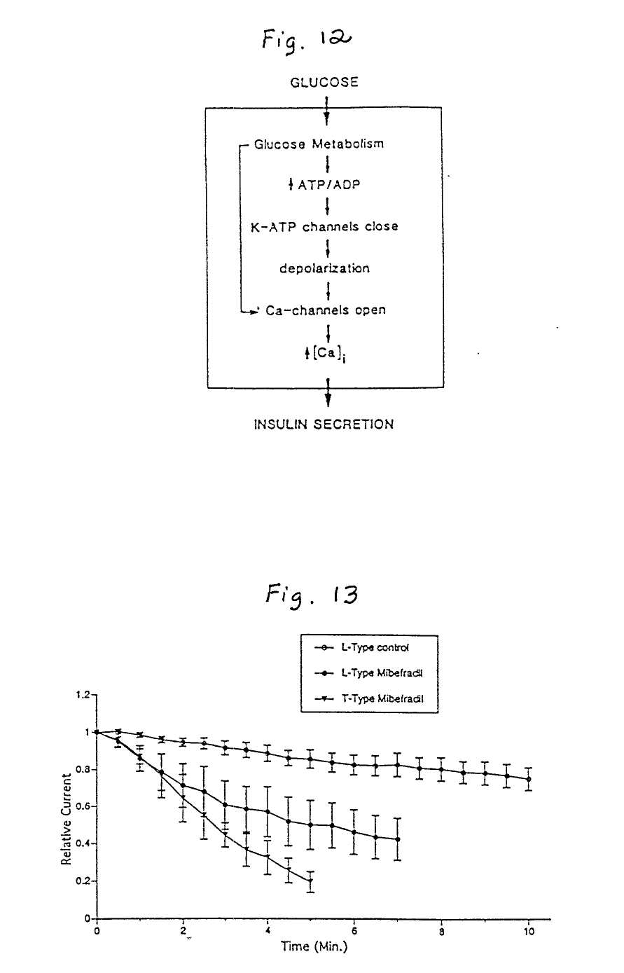

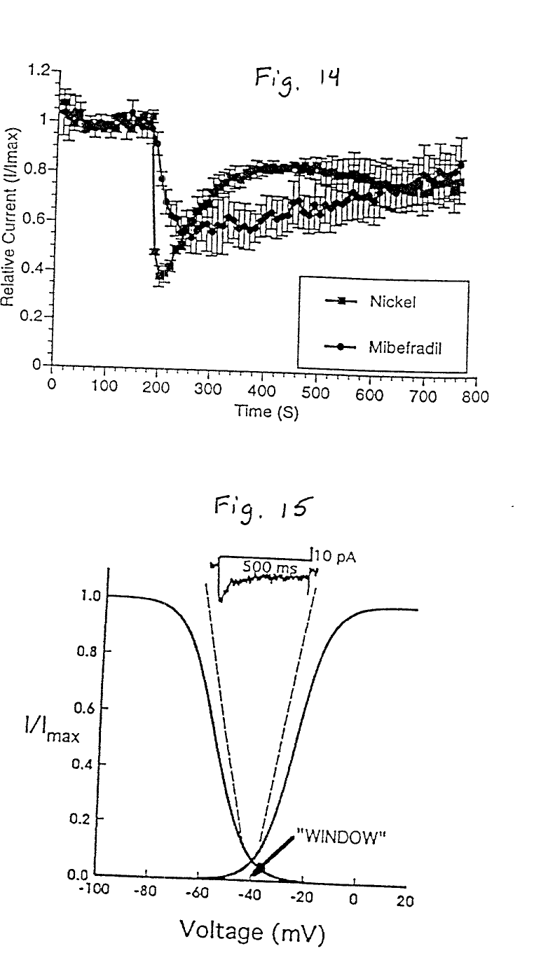

In β-cells, T-type Ca²⁺ channels could mediate a small, but sustained, Ca²⁺ influx by means of their unique “window” current at voltages near resting membrane potentials. Like other voltage-regulated channels, T-type Ca²⁺ channels are opened and closed depending upon the potentials across the cell membranes. This voltage dependency is illustrated in FIG. 15. The activation and inactivation curves represent the percentage of the channels in either open or closed states over a range of voltages. Unlike most of the voltage-dependent Na⁺ channels or L-type Ca²⁺ channels, the activation and inactivation curves of T-type Ca²⁺ channels overlap at the certain range of low voltages (i.e. window). In other words, there is a small portion of T-type Ca²⁺ channels that stay in non-inactivated states in this voltage range. The data in FIG. 15 was obtained from experiments conducted under 10 mM external Ca²⁺ condition, which shifted the window current about 10 mV toward positive voltage due to the surface charge effects of divalent ions on the channels.

The existence of a window current provides a negative feedback regulation of [Ca²⁺]ᵢ in β-cells. When cells are under an unhealthy condition, they may be slightly depolarized to activate window current, which elevates the basal [Ca²⁺]ᵢ to protect the cells from further Ca²⁺ influx through the L-type Ca²⁺ channels. This process is reversible if the membrane potential is reset to the normal resting potential (−70 mV).

Mibefradil Regulates Basal [Ca²⁺]ᵢ in Pancreatic β-Cells:

The data herein demonstrates the roles of T-type calcium currents in modulating basal [Ca²⁺]ᵢ in INS-1 cells (FIG. 8). [Ca²⁺]ᵢ was directly measured by the ratio of fluorescence excitations at Ca²⁺-bound (380 nm) to unbound (340 nm), and then the ratio was converted to the calcium concentration. The bath solution contained 10 mM NaCl, 4 mM KCl, 2 mM CaCl₂, and 2 mM MgCl₂. In a single cell exhibiting fluctuating basal [Ca²⁺]ᵢ with an average value near 150 nM, administering 1 μM mibefradil into the chamber immediately lowered the basal calcium. This data shows the T-type calcium currents participate in regulating the mean basal [Ca²³⁰ ]ᵢ in cultured β-cells.

Mibefradil Regulates Basal Insulin Secretion:

The activation of T-type Ca²⁺ channel at low voltage near the resting membrane potential of pancreatic β-cells suggests that the channels are responsible for the Ca²⁺ influx required for insulin secretion under non-stimulus conditions. The NIT-1 cell line was chosen to demonstrate the effect of mibefradil on the basal insulin secretion. NIT-1 is a cell line derived from the β-cells of non-obese-diabetic mouse. This cell line expressed high levels of T-type Ca²⁺ current. The data herein shows that 5 μM mibefradil reduced the basal insulin secretion to less than 40′ of control (FIG. 17), indicating this drug is able to lower the high basal insulin secretion level seen during the earlier stage of NIDDM.

Spontaneous Elevation of [Ca²⁺]ᵢ:

To demonstrate that T-type Ca²⁺ channels play an important role in calcium entry under non-stimulatory conditions, and therefore regulate basal [Ca²⁺]ᵢ, spontaneous elevations of intracellular free calcium concentration was detected with the Fluo-3 μM fluorescent imaging method. NIT-1 cells were cultured in medium containing 3.3 mM glucose and preloaded with 2.5 μM Fluo-3 μM. The numbers of spontaneous calcium elevated cells were counted and compared to the total cells being used for a 10 minute observation period. 10 μM NiCl₂ inhibited 90% of spontaneous elevation of basal Ca²⁺.

The cellular mechanism of the spontaneous elevation of intracellular Ca²⁺ was investigated with the epi-fluorescence measurement method. Some INS-1 cells were observed to exhibit transient spontaneous elevations of [Ca²⁺]ᵢ, “Calcium spikes”, under non-stimulatory conditions. The Role of T-type Ca²⁺ channels in this spontaneous process was examined as well. In a single cell with spontaneous calcium spike activity (FIG. 17), NiCl₂ (30 μM) reduced the frequency of spontaneous calcium spikes immediately. This result suggests that either the T-type Ca²⁺ channels alone or together with the L-type Ca² channels are responsible for the transient spontaneous elevation of [Ca²⁺]ᵢ, under conditions where no glucose is present. These spontaneous calcium spikes may contribute to basal insulin secretion and control of basal [Ca²⁺]ᵢ.

However, neither mibefradil nor NiCl₂ exhibited their effect on basal [Ca²⁺]ᵢ in all of the β-cells. It was observed that only those cells which had relatively higher initial basal [Ca²⁺]ᵢ will respond to the T-type Ca²⁺ channel antagonists (FIG. 18). Whereas those cells with lower initial basal [Ca²⁺]ᵢ had no or less response to the T type Ca²⁺ channel antagonists. This result indicates that T type Ca²⁺ channel antagonists may selectively act on the cells with high basal [Ca²⁺]ᵢ and bring it back to normal, by inhibiting the window current.

### EXAMPLE IV

### Action on Pancreatic β-Cells

T type Ca²⁺ may play two pathological roles in NIDDM. At the earlier stage, the NIDDM patients exhibit hyperinsulinemia and β-cell hyperexcitability. This may, at least in part, be due to increased activity of T type Ca²⁺ channel in β-cells. At the more developed NIDDM stage, over-expressed T type Ca²⁺ channel and membrane depolarization resulted from reduced generation of ATP, and may set up a window current in β-cells that causes chronic elevation of basal Ca²⁺ in the β-cells. The elevated basal Ca²⁺ will reduce the L-type Ca²⁺ activity and glucose induced insulin secretion.

It has been shown that mibefradil prevented and reversed development of hyperinsulinemia in rat. This result indicates this drug is a valuable candidate for the treatment of earlier stage NIDDM or for preventing NIDDM in the potential patients.

A series of experiments were conducted with INS-1 cells to show that T type Ca²⁺ facilitated insulin secretion by enhancing the general excitability of pancreatic β-cells. Particularly, activation of T type Ca²⁺ channels will increase the firing frequency of the depolarizing spikes mediated by opening L type Ca²⁺ channels (FIG. 19A). Activation of T type Ca²⁺ channel will also decrease the time of developing action potential elicited by up-threshold depolarizations (FIG. 19B).

To further establish that T type Ca²⁺ current enhances β-cells excitability, 100 μM NiCl₂ was administered to effectively block T type Ca²⁺ channels. In contrast to control experiments, NiCl₂ caused a delay in the onset of an action potential and a decrease in number of action potentials.

To directly demonstrate the role of T type Ca²⁺ current in glucose-induced insulin secretion, INS-1 cells were incubated with 11.1 mM glucose and variable concentrations of NiCl₂, and insulin release was measured. NiCl₂ reduced insulin secretion in a dose-dependent manner (FIG. 20A). On the other hand, clonal insulin secreting cells (HIT-T15, which did not consistently exhibit T type Ca²⁺ current) were not affected by NiCl₂ (FIG. 20B).

These results show that T type Ca²⁺ channels play an important role in β-cell excitability and antagonists of T type Ca²⁺ channels (such an NiCl₂) will effectively reduce the excitability of β-cells.

Although T type Ca²⁺ channels facilitate insulin secretion by enhancing general excitability of β-cells, the function of T type Ca²⁺ channels is a doubled-edged sword. Under the condition of over-expressed T type Ca²⁺ channel in β-cells, the function of the window current will become dominant and result in an elevation of basal Ca²⁺ . High [Ca²⁺]ᵢ may cause impairment of insulin release by inactivating L type Ca²⁺ channels.

L-Type Ca²⁺ Channels are Partially Inactivated by [Ca²⁺]ᵢ in Non-Stimulus Condition in β-Cells:

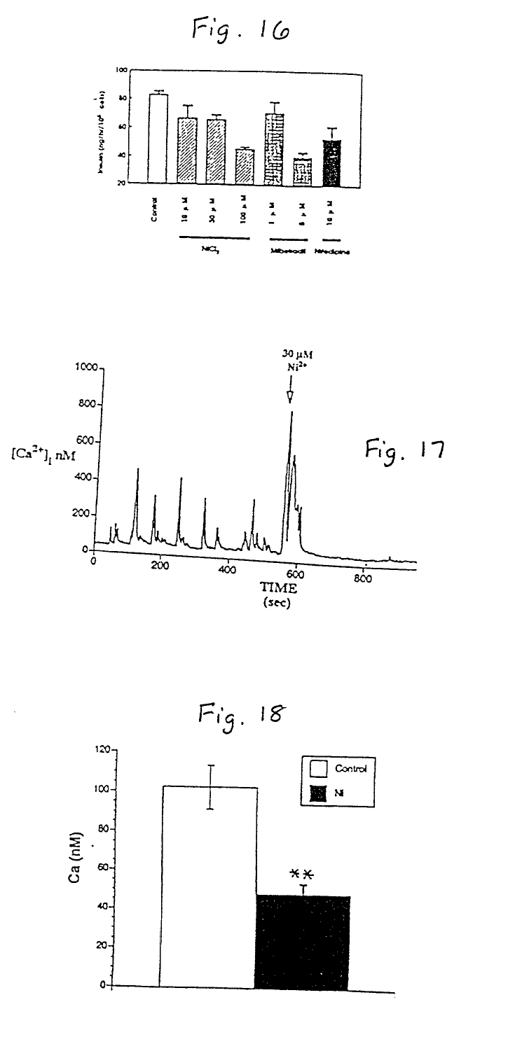

Upon establishment of a whole-cell patch, within the first five minutes, the L type Ca²⁺ current “runs-up”, as the magnitude of the peak current increases over time in INS-1 cells (FIG. 21). This phenomenon is a universal feature in these cells under the recording conditions used. The pipette solutions contained no ATP but did contain high concentrations of the calcium chelating agents BAPTA and EGTA. When the pipette solution contained high Ca²⁺, this run-up does not occur. Instead, a rapid run down occurs. The “run-up” phenomenon is likely due to calcium chelation inside the cells. T type Ca²⁺ currents do not show this effect.

Intracellular Perfusion Patch Clamp Experiments Demonstrated That Basal [Ca²⁺]ᵢ Regulates L Type Ca²⁺ Current Amplitude in INS-1 Cells:

Intracellular perfusion of a solution containing high Ca²⁺ (FIG. 9A) causes a substantial reduction in the L type Ca²⁺ current. L type Ca²⁺ currents were elicited by a voltage step to +10 mV from a holding potential of −80 mV. The [Ca²⁺]ᵢ was measured directly using fura-2 ratiometric fluorescence. The effect of a high [Ca²⁺]ᵢ (272 nM) on the IV relationship is shown in FIG. 9B. Perfusing in high [Ca²⁺]ᵢ, substantially reduces the high voltage current component, but does not affect the low current component. The high [Ca²⁺]ᵢ caused a shift in peak current to negative voltages, and Ca²⁺ currents were enhanced at negative voltages. This effect seemed to result in a potentiation of the T type Ca²⁺ current (FIG. 9D)). Slow deactivating T type Ca² currents showed a shift in activation upon perfusion of high [Ca²⁺]ᵢ. This may account for the shift in IV. Various concentrations of [Ca²⁺]ᵢ regulated the activity of L type Ca²⁺ channels (FIG. 9C). Perfusing a low [Ca²⁺]ᵢ from an existing high [Ca²⁺]ᵢ (632 nM to 0 nM) caused an increase in the L type Ca²⁺ current over time, however perfusing in high [Ca²⁺]ᵢ (0 nM to 272 nM and 0 nM to 632 nM) inhibits the L type Ca²⁺ current over time. The levels of [Ca²⁺]ᵢ therefore have regulatory effects on both the L type Ca²⁺ current and T type Ca²⁺ current, with [Ca²⁺]ᵢ having significant feedback regulation on the L type Ca²⁺ current.

Effect of Basal [Ca²⁺]ᵢ on Ca²⁺ Influx:

The effect of basal [Ca²⁺]ᵢ on Ca²⁺ influx was examined using the Ca²⁺ dye indicator fura-2 and fluorescence measurements. Voltage-dependent Ca²⁺ influx in a single cell was obtained by perfusion of an osmotically balanced solution containing 50 mM KCl into the recording chamber. Voltage-dependent increases in [Ca²⁺]ᵢ occur primarily through nifedipine sensitive Ca²⁺ channels. The resting basal [Ca²⁺]ᵢ in INS-1 cells was approximately 60-80 nM under the experimental conditions. [Ca²⁺]ᵢ was determined by a standard curve obtained from a fura-2 calcium imaging kit (Molecule Probes). The empirical K_{d} obtained for calcium binding to fura-2 in the system was 296±20 nM. When basal [Ca²⁺]ᵢ remains low, subsequent voltage stimulation with 50 KCl induces rapid and large calcium influx into the cell and these calcium changes are stereotyped upon repetitive stimulation when basal calcium is restored (FIG. 10). In this experiment, following the 50 KCl depolarization, the cell was repolarized by perfusion of the original 5 mM KCl solution. After repolarization, basal [Ca²⁺]ᵢ slowly reset and then a second 50 KCl depolarization induced a similar [Ca²⁺]ᵢ transient. When the basal calcium is not allowed to reset, a defect in the second voltage induced calcium transient occurs (FIG. 11). In this experiment, after repolarization, the second depolarization was applied before basal [Ca²⁺]ᵢ could return to its original value, and thus, the [Ca²⁺]ᵢ transient is substantially reduced. These findings suggest that basal [Ca²⁺]ᵢ plays a prominent role in the regulation of voltage dependent Ca²⁺ influx in INS-1 cells. Therefore effectors of basal [Ca²⁺]ᵢ will have important impact on the amount of calcium that can enter the cell.

Streptozotocin Induced High Basal [Ca²⁺]ᵢ Inhibits KCl Stimulated Ca²⁺ Influx:

To reiterate the importance of basal [Ca²⁺]ᵢ on voltage stimulated Ca²⁺ influx, basal [Ca²⁺]ᵢ in INS-1 cells was artificially enhanced by pretreating the cells with the toxicant, streptozotocin. Though it is know that streptozotocin induces DNA strand breaks, it has also been shown to induce Ca²⁺ channel activity in β-cells. The data shows that pretreating cells with 5 mM streptozotocin for 1 hour, followed by 3 hour recovery, causes a two-fold increase in basal calcium (FIG. 22). These cells when stimulated by 50 KCl had reduced calcium influx compared to control cells.

### EXAMPLE V

### Inhibition of T Type Calcium Channel With Mibefradil Metabolite

It has been shown that mibefradil (Ro 40-5967) exerts a selective inhibitory effect on T-type Ca²⁺ currents, although at higher concentrations it can antagonize high voltage-activated Ca²⁺ currents. The action of mibefradil on Ca²⁺ channels is use- and steady state-dependent and the binding site of mibefradil on L-type Ca²⁺ channels is different from that of dihydropyridines. By using conventional whole-cell and perforated patch-clamp, mibefradil is shown to have an inhibitory effect on both T- and L-type Ca²⁺ currents in insulin-secreting cells. However, the effect on L-type Ca²⁺ currents was time-dependent and poorly reversible in perforated patch experiments. Using mass spectrometry it was demonstrated that mibefradil was trapped inside cells and furthermore, a metabolite of mibefradil was detected. Intracellular application of this metabolite selectively blocked the L-type Ca²⁺ current whereas mibefradil exerted no effect. This study shows that mibefradil permeates into cells and is hydrolyzed to a metabolite that blocks L-type Ca²⁺ channels specifically by acting at the inner side of the channel.

Whole-cell patch clamp and a bath perfusion system were first used to establish the dose-dependent inhibition of mibefradil on both T- and L-types of Ca²⁺ currents. The T-type Ca²⁺ current was measured at −30 mV when the membrane was held at −90 mV and the L-type current was measured at +20 mV when the membrane was held at −40 mV. The currents were measured twice at each concentration of mibefradil with 2 min in between measurements. The dose dependent inhibition of T-type Ca²⁺ current is shown in FIG. 3A. The 50% inhibitory concentration (IC₅₀) was 865 nM. No time-dependent inhibition was observed. In contrast, the inhibition of L-type Ca²⁺ currents could not be fitted with a one-to-one binding curve (FIG. 3B). Administration of 1 μM mibefradil progressively reduced L-type Ca²⁺ current up to 70% of the beginning amplitude after 10 minutes (n=4), which indicated that a more complicated pharmacological mechanism was involved in the action of mibefradil on the L-type Ca²⁺ currents.

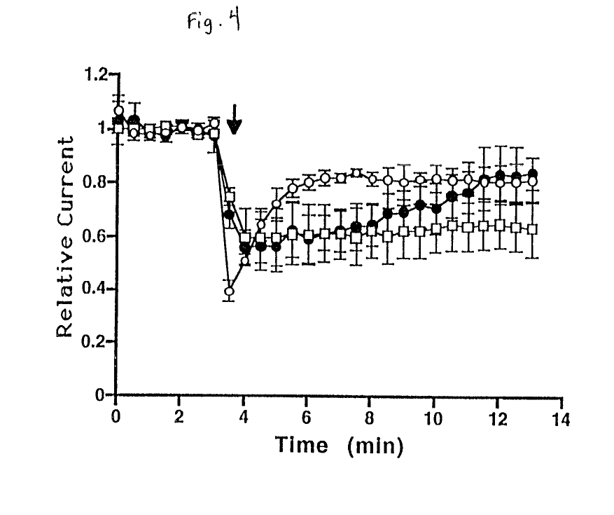

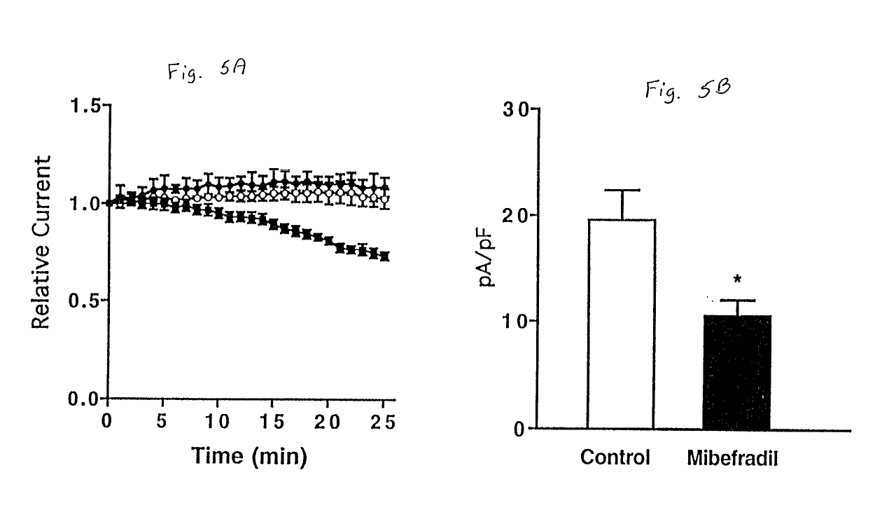

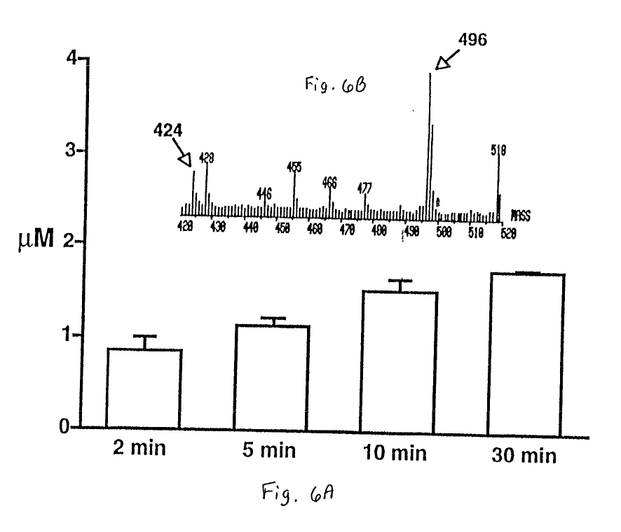

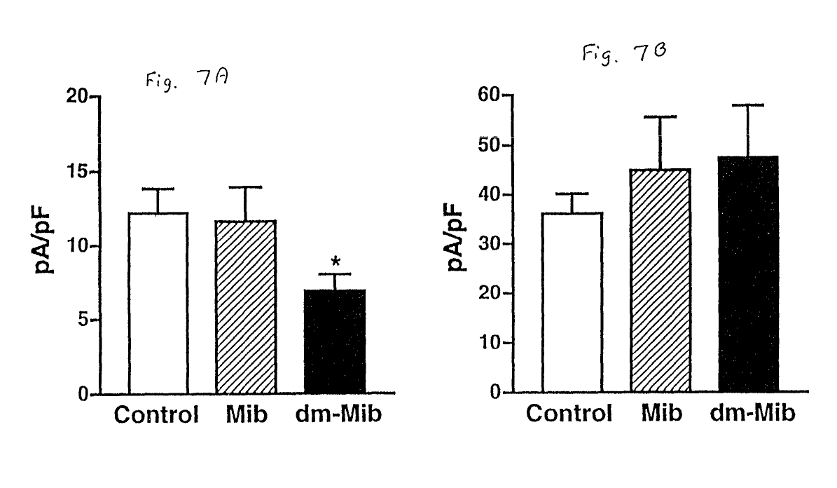

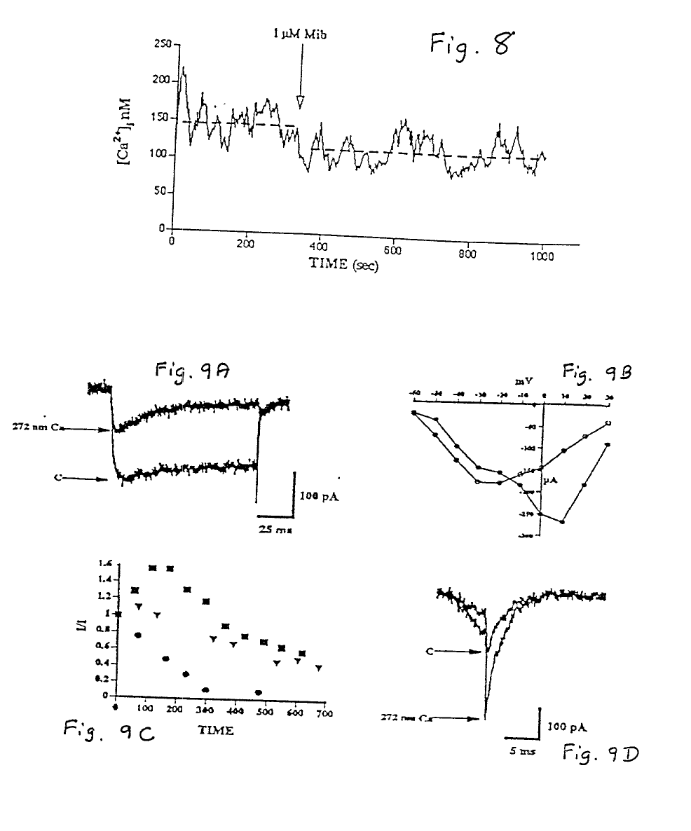

A drug diffusing system was then used to test the reversibility of the antagonism of T- and L-type Ca²⁺ currents by mibefradil. Small volumes (approximately 2 μl) of drugs were delivered in close proximity to the recording cell with a quartz capillary positioned by a micromanipulator. After administration, drugs diffused throughout the entire recording chamber containing 2 ml of bath solution. This drug diffusing system was used to test the reversibility of 30 μM of NiCl₂ on the T-type Ca²⁺ currents (FIG. 4). The amplitude of T-type current was rapidly reduced to 40% and gradually returned to 80% of the initial level within 3 minutes. Using this system, it was found that the inhibition of mibefradil on the T-type Ca²⁺ current was clearly reversible. In contrast, the inhibition of the L-type Ca²⁺ current was poorly reversible (FIG. 4).

The poor reversibility and time-dependent inhibition of the L-type Ca²⁺ current by mibefradil suggested that this drug may have an accumulation effect over time. This hypothesis was tested by applying a very low dose of mibefradil on cells and recording the L-type Ca²⁺ currents for a long time in the perforated patch-clamp configuration. As shown in FIG. 5A, after 25 minutes of 10 nM mibefradil administration, the relative currents were reduced to 70%, whereas the currents remained unchanged for control patches. Incubation of cells with 10 nM mibefradil for two hours resulted in further reduction of current densities as recorded by perforated patches (FIG. 5B). At a concentration of 10 nM, mibefradil exhibited no long-term effect on the T-type Ca²⁺ current.

To test the hypothesis that mibefradil may permeate through the cell membrane to the cytoplasm and be trapped inside cells, the presence of mibefradil was examined in cells pre-incubated with 20 μM of mibefradil using mass spectrometry. After 3 washes, mibefradil (peaked at 496 MW) was still detected in cells (FIG. 6B). The concentration of intracellular mibefradil after one minute incubation was 3.18±0.78 μM (n=3). The localization of mibefradil in cells was examined by measuring the concentration of mibefradil in the pellets and supernatants after lysis of the cells. Most of the mibefradil (92%) was detected in the supernatants and 0% was found in the pellets after washing cells with methanol, indicating that mibefradil was trapped in the cytoplasm. In addition, a peak (MW=423) was detected which represented a hydrolyzed metabolite of mibefradil, Des-methoxyacetyl mibefradil (dm-mibefradil), which is a major metabolite as documented previously (Wiltshire et al. 1992). By varying the time of pre-incubation, it was found that dm-mibefradil accumulated inside the cells in a time-dependent manner (FIG. 6A). This accumulation is consistent with the concept that dm-mibefradil has lower membrane permeability than its precursor mibefradil.

It was then tested whether or not mibefradil or dm-mibefradil inhibits L- or T-type Ca²⁺ currents from inside of cells. Both L- and T-type currents were measured in the whole-cell patch clamp configuration when 1 μM of mibefradil or dm-mibefradil was included in the pipette solution. As shown in FIGS. 7A and 7B, intracellular application of 1 μM mibefradil did not have inhibitory effects on either L-type or T-type Ca²⁺ currents, whereas the same concentration of dm-mibefradil specifically blocked the L-type Ca²⁺ current. As the bath solution contained no drug in this series of experiments, the inhibitory effect of dm-mibefradil is considered to be acting on the inside domain of L-type Ca²⁺ channels.

The inhibitory effect of dm-mibefradil on T-type Ca²⁺ currents was similar to the effect of mibefradil when it was applied in the bath solution, suggesting that the methoxyacetyl group of mibefradil does not play a key role in binding to the extracellular receptor site of T-type Ca²⁺ channel protein. However, this methoxyacetyl group is necessary for blocking L-type Ca²⁺ channel from the inside of cells, indicating a modification in the methoxyacetyl group of mibefradil can result in a more selective antagonist of T-type Ca²⁺ channels.

### EXAMPLE VI

### LVA Ca²⁺ Current Mediates Cytokine-Induced Pancreatic β-Cell Death

Insulin-dependent diabetes mellitus is characterized by the selective destruction of pancreatic β-cells. Chronic treatment with cytokines induced a low voltage-activated (LVA) Ca²⁺ current in mouse β-cells. The concomitant increase in the basal cytoplasmic free Ca²⁺ concentration ([Ca²⁺]ᵢ) was associated with DNA fragmentation and cell death. Antagonists of LVA Ca²⁺ channels prevented this elevation of basal [Ca²⁺]ᵢ and DNA fragmentation, and reduced the percentage of cell death. Exposure to cytokines did not affect the profile of Ca²⁺ currents or basal [Ca²⁺]ᵢ in glucagon-secreting α-cells. An increased Ca²⁺ signal through LVA Ca²⁺ channels may thus be a key feature in cytokine-induced β-cells destruction.

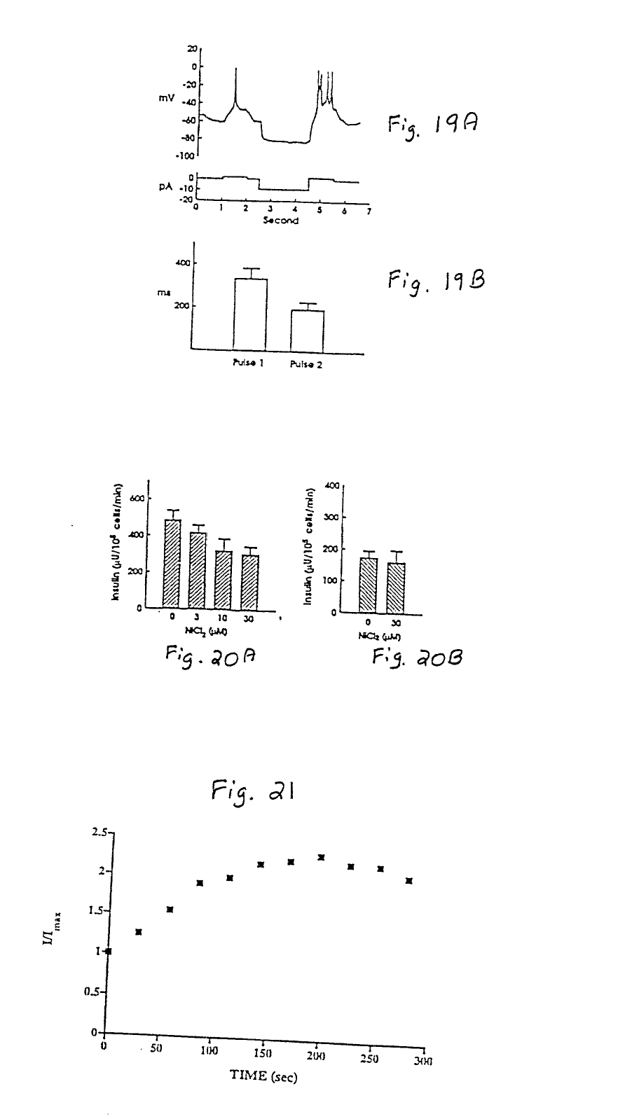

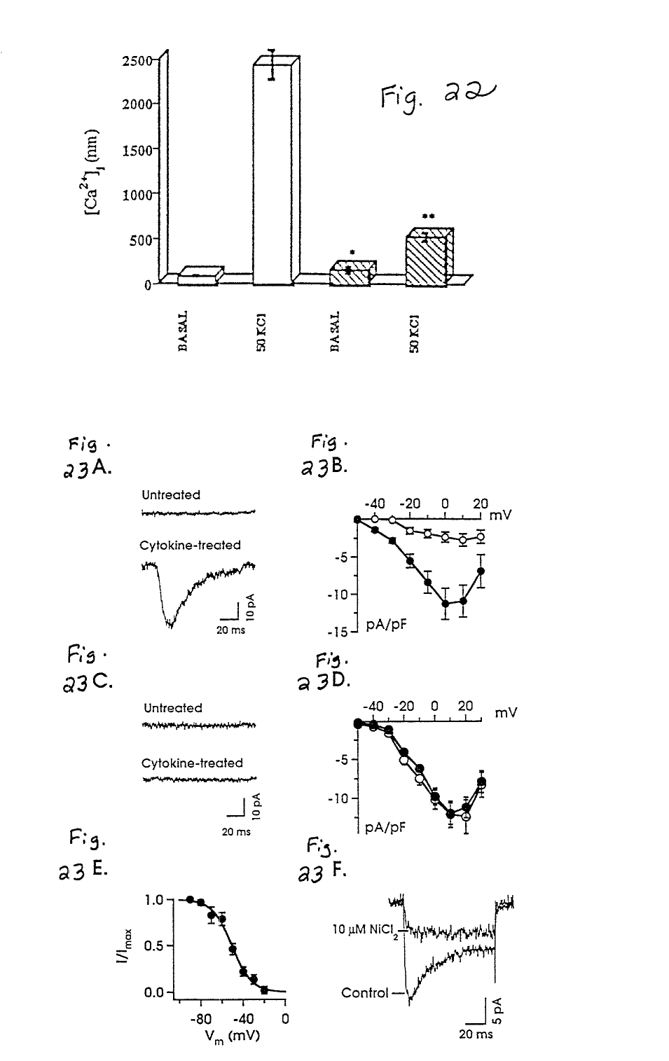

The effects of chronic cytokine treatment on the voltage-sensitive Ca²⁺ currents in primary cultured mouse islet cells was examined. After treatment with IL-1β (25 U/ml) and IFNγ (300 U/ml) for 6 h, an LVA Ca²⁺ current was induced in these cells (FIG. 23A). This current was present in 48% of cytokine-treated mouse islet cells. No such current was observed when the cells were treated with either IL-1β or IFNγ alone. Experiments were conducted at different times recording LVA Ca²⁺ currents induced by cytokines, and the results indicate that no further increase in current density occurs even after treatment for 48 h. This LVA current has not been observed in non-treated cells. The steady state inactivation curve of the cytokine-induced LVA Ca²⁺ currents displayed a low voltage property (FIG. 23E) similar to the inactivation curve of the LVA currents in NOD mouse islets cells. This current was also blocked by NiCl₂ (10 μM; n=4; FIG. 23F). It has been reported that low concentration of NiCl₂ selectively block LVA current, a profound increase in Ca²⁺ current density was observed over the voltages between −20 and 20 mV. These high voltage-activated Ca²⁺ currents are nifedipine sensitive currents (completely blocked by 10 μM nifedipine), and the increase in this current density is similar to the increased L type Ca²⁺ current density observed after treatment of β-cells with serum from IDDM patients.

As α-cells are more resistant to the toxic effects of cytokines than β-cells, the effects of cytokines on the Ca²⁺ currents in a glucagon-secreting cell line (α-TC1) was also examined. This cell line, like α-cells, is more resistant to the cytotoxic effect of cytokines. Treatment of α-TC1 cells with IL-1β and IFNγ failed to induce LVA Ca²⁺ currents and did not alter the current density (FIGS. 23C and 23D). Therefore, the induction of LVA Ca²⁺ currents and increased Ca²⁺ current density observed after chronic treatment with cytokines showed specificity for β-cells.

LVA Ca² channels are activated at low membrane potentials. This unique feature may allow then to regular [Ca²⁺]ᵢ under nonstimulatory conditions. Indeed, basal [Ca²⁺]ᵢ in cytokine-treated cells was approximately 3-fold higher than in nontreated cells (FIG. 24A). This increase in basal [Ca²⁺]ᵢ was blocked by NiCl₂ (10 μM), but not by the L type Ca²⁺ channel antagonist, nifedipine (10 μM). Cytokines failed to increase basal [Ca²⁺]ᵢ in α-TC1 cells (FIG. 24B). These results suggest that Ca²⁺ influx through LVA Ca²⁺ channels is responsible for the cytokine-induced elevation in basal [Ca²⁺]ᵢ in β-cells.

It has been shown that cytokines induce apoptosis in human pancreatic islet cells. Apoptosis is also the mode of cell death in the development of IDDM in the NOD mouse and in multiple low dose streptozotocin-induced IDDM in the mouse, and is involved in β-cell destruction. As a marker of apoptosis, DNA fragmentation has been reported to precede β-cell lysis.

β-TC3 cells, a mouse β-cells line, were used to demonstrate the role of LVA Ca²⁺ channels in cytokine-mediated DNA fragmentation. The LVA Ca²⁺ current density was first examined before and after cytokine treatment. The LVA Ca²⁺ current (at Vₘ=−30 mM) in β-TC3 cells was increased from 1.86±0.33 (pA/pF; n=30) to 3.45±0.47 (pA/pF; n=10) after treatment with cytokines (25 U/ml IL-1β, 100 U/ml IFNγ, and 100 U/ml TNFα) for 25 h. This indicates that the LVA Ca²⁺ current in β-TC3 cells is regulated by cytokines, as seen in mouse islet cells. As shown in FIG. 24, cytokine-induced DNA fragmentation displayed a ladder pattern of oligonucleosomal fragments. The three LVA Ca²⁺ channel blockers, NiCl₂, amiloride, and mibefradil, all independently prevented cytokine-induced DNA fragmentation. In contrast, nifedipine had not inhibitory effect on DNA fragmentation induced by cytokines. This experiment has been repeated in β-TC3 cells (n=2) as well as in NIT-1 cells (n=3), a β-cells line derived from NOD mice, and the same results were obtained.

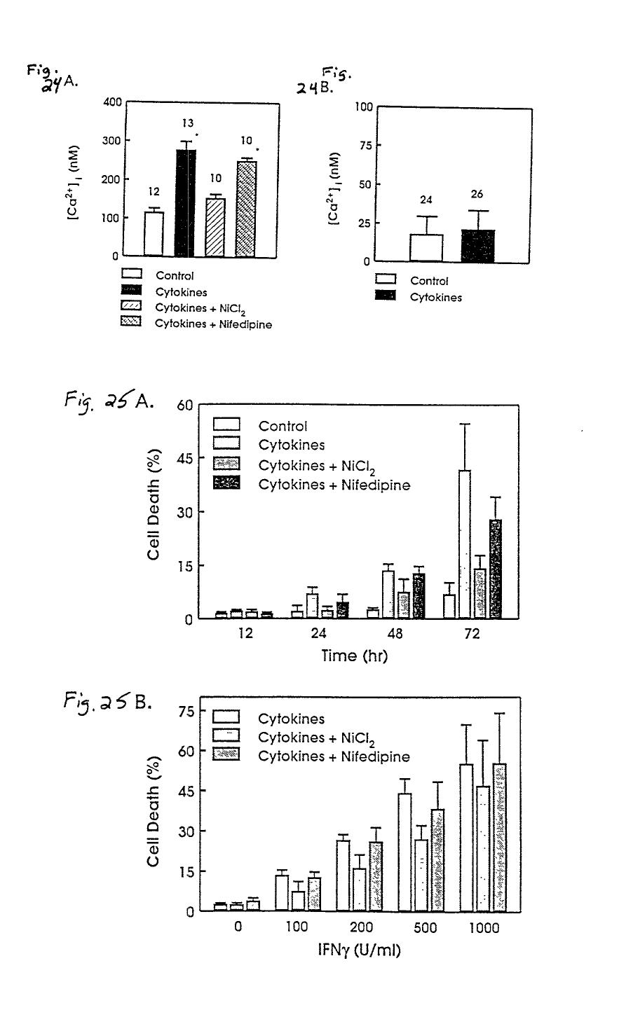

The function of LVA Ca²⁺ channels in cytokine-mediated cell death in β-TC3 cells was then examined. Many cells died when the medium contained 25 U/ml IL-1β, 100 U/ml IFNγ, and 100 U/ml TNFα; however, NiCl₂ (20 μM) effectively reduced the β-cells killing potency of cytokines in both a time- and dose-dependent manner (FIGS. 25A and 25B, respectively). In contrast, nifedipine did not exhibit a protective effect. Similar results were obtained from an experiment conducted in NIT-1 cells with mibefradil, which also reduced β-cells death induced by cytokines. These results demonstrate that LVA Ca²⁺ channels enhance the vulnerability of β-cells to the cytotoxic effects of cytokines.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

### REFERENCES

Asfari, M., et al., Endocrinology 130:167-178 (1992).

Bayer, E. A., et al., Meth Enzym 62:308 (1979).

Bhattacharjee, A., et al., Endocrinology 138:3735-3740 (1997).

Boyd, A. E. III, Current Concepts, The Upjohn Company, Kalamazoo, Mich. (1991).

Campbell, A. M., Monoclonal Antibody Technology: Laboratory Techniques in Biochemistry and Molecular Biology, Elsevier Science Publishers, Amsterdam, The Netherlands (1984).

Capecchi, M., Cell 22:479-488 (1980).

Catterall, W. A., Science 242:50-61 (1988).

Catterall, W. A., Science 253:1499-1500 (1991).

Chomczynsk, P., et al., Anal. Biochem. 162:156-157 (1987).

Chrisey, L., et al., Antisense Research and Development 1(1):57-63 (1991).

Christoffersen, R. E. and Marr, J. J., Journal of Medicinal Chemistry 38(12):2023-2037 (1995).

Davalli, A. M., et al., J Endocrinology 150:195-203 (1996).

Engval, E., et al., Immunol 109:129 (1972).

Goding, J. W., J Immunol Meth 13:215 (1976).

Han, L., et al., Proc Natl Acad Sci USA 88:4313-4317 (1991).

Hiriart, M. and Matteson, D. R., J Gen Physiol 91:145-159 (1988).

Innis, et al., PCR Protocols, Academic Press, San Diego, Calif. (1990).

Kato, S., et al., Metabolism 43:1395-1400 (1994).

Kato, S., et al., J Clin Invest 97:2417-2425 (1996).

Keahey, H. H., et al., Diabetes 38:188-193 (1989).

Klein, T. M., et al., Nature 327:70-73 (1987).

Lutz, et al., Exp Cell Res 175:109-124 (1988).

Mannino, R. J. and Gould-Fogerite, S., BioTechniques 6:682-690 (1988).

Miller, L. K., Bioessays 11:91-95 (1989).

Perez-Reyes, E., et al., Nature 391:896-900 (1998).

Rossi, J. J., et al., AIDS Research and Human Retroviruses 8(2):183-189 (1992).

Rossi, J. J., British Medical Bulletin 51(1):217-225 (1995).

Sambrook et al., Molecular Cloning: A Laboratory Manual, 2d Edition, Cold Spring Harbor Laboratory Press, Cold Spring Harbor, N.Y. (1989).

Sarver, N., et al., Science 247:1222-1225 (1990).

Seino, S., et al., Proc Natl Acad Sci USA 89:584-588 (1992).

Shigekawa, K. and Dower, W. J., BioTechniques 6:742-751 (1988).

Stea, A., et al., In: Ligand and voltage-gated ion channels. pp113-151, ed. R. Alan North, CRC Press, Boca Raton (1995).

Sternberger, L. A., et al., J Histochem Cytochem 18:315 (1970).

St. Groth, et al., J Immunol Methods 35:1-21 (1980).

Vague, P. and Moulin, J. P., Metabolism 31:139-144 (1982).

Wiltshire, H. R., et al., Xenobiotica 22:837-857 (1992).

Wang, L., et al., Diabetes 45:1678-1683 (1996).

Yaney, G. C., et al., Mol Endocrinol 6:2143-2152 (1992).

What is claimed is:

1. An isolated nucleic acid molecule encoding a pancreatic T-type calcium channel.

2. The isolated nucleic acid molecule of claim 1 wherein said nucleic acid is deoxyribonucleic acid.

3. The isolated nucleic acid molecule of claim 2 wherein said deoxyribonucleic acid is cDNA.

4. The isolated nucleic acid molecule of claim 3 wherein said nucleic acid molecule has a nucleotide sequence as shown in SEQ ID NO:1.

5. The isolated nucleic acid molecule of claim 1 wherein said nucleic acid molecule encodes an amino acid sequence as shown in SEQ ID NO:2.

6. The isolated nucleic acid molecule of claim 1 wherein said nucleic acid is ribonucleic acid.

7. The isolated nucleic acid molecule of claim 6 wherein said ribonucleic acid is mRNA.

8. An antisense nucleic acid molecule complementary to at least a portion of the mRNA of claim 7.

9. A cell comprising the antisense nucleic acid molecule of claim 8.

10. An expression vector comprising the antisense nucleic acid molecule of claim 8.

11. The expression vector of claim 10 wherein the expression vector is selected from the group consisting of a plasmid and a virus.

12. A cell comprising the expression vector of claim 10.

13. A method of decreasing expression of a pancreatic T-type calcium channel in a host cell, said method comprising introducing the antisense nucleic acid molecule of claim 8 into the cell, wherein said antisense nucleic acid molecule blocks translation of said mRNA so as to decrease expression of said pancreatic T-type calcium channel in said host cell.

14. A ribozyme having a recognition sequence complementary to a portion of the mRNA of claim 7.

15. A cell comprising the ribozyme of claim 14.

16. An expression vector comprising the ribozyme of claim 14.

17. The expression vector of claim 16 wherein the expression vector is selected from the group consisting of a plasmid and a virus.

18. A cell comprising the expression vector of claim 16.

19. A method of decreasing expression of a pancreatic T-type calcium channel in a host cell, said method comprising introducing the ribozyme of claim 14 into the cell, wherein expression of said ribozyme in said cell results in decreased expression of said pancreatic T-type calcium channel in said cell.

20. A cell comprising the nucleic acid molecule of claim 1.

21. An expression vector comprising the nucleic acid molecule of claim 1.

22. The expression vector of claim 21 wherein said expression vector is selected from the group consisting of a plasmid and a virus.

23. A cell comprising the expression vector of claim 21.

24. A method of increasing expression of pancreatic T-type calcium channel in a host cell, said method comprising:
introducing the nucleic acid molecule of claim 1 into the cell; and
allowing said cell to express said nucleic acid molecule resulting in the production of pancreatic T-type calcium channel in said cell.

25. A method of screening a substance for the ability of the substance to modify T-type calcium channel function, said method comprising:
introducing the nucleic acid molecule of claim 1 into a host cell;
expressing said pancreatic T-type calcium channel encoded by said nucleic acid molecule in the host cell;
exposing the cell to a substance; and
evaluating the exposed cell to determine if the substance modifies the function of the T-type calcium channel.

26. The method of claim 25 wherein said evaluation comprises monitoring the expression of T-type calcium channel.

27. A method of obtaining DNA encoding a pancreatic T-type calcium channel, said method comprising:
selecting a DNA molecule encoding a pancreatic T-type calcium channel, said DNA molecule having a nucleotide sequence as shown in SEQ ID NO:1;
designing an oligonucleotide probe for a pancreatic T-type calcium channel based on SEQ ID NO:1;
probing a genomic or cDNA library of an organism with the oligonucleotide probe; and
obtaining clones from said library that are recognized by said oligonucleotide probe, so as to obtain DNA encoding a pancreatic T-type calcium channel.

28. A method of obtaining DNA encoding a pancreatic T-type calcium channel, said method comprising:
selecting a DNA molecule encoding a pancreatic T-type calcium channel, said DNA molecule having a nucleotide sequence as shown in SEQ ID NO:1;
designing degenerate oligonucleotide primers based on SEQ ID NO:1; and
utilizing said oligonucleotide primers in a polymerase chain reaction on a DNA sample to identify homologous DNA encoding a pancreatic T-type calcium channel in said sample.

29. An isolated nucleic acid molecule encoding a pancreatic T-type calcium channel, said nucleic acid molecule encoding a first amino acid sequence having at least 90% amino acid identity to a second amino acid sequence, said second amino acid sequence as shown in SEQ ID NO:2.

30. A DNA oligomer capable of hybridizing to the nucleic acid molecule of claim 1.

31. A method of detecting presence of a pancreatic T-type calcium channel in a sample, said method comprising:
contacting a sample with the DNA oligomer of claim 30, wherein said DNA oligomer hybridizes to any of said pancreatic T-type calcium channel present in said sample, forming a complex therewith; and
detecting said complex, thereby detecting presence of a pancreatic T-type calcium channel in said sample.

32. The method of claim 31 wherein said DNA oligomer is labeled with a detectable marker.

33. An isolated pancreatic T-type calcium channel protein.

34. The pancreatic T-type calcium channel protein of claim 33 wherein said pancreatic T-type calcium channel protein is encoded by a nucleotide sequence as shown in SEQ ID NO:1.

35. The pancreatic T-type calcium channel protein of claim 33 wherein said pancreatic T-type calcium channel protein is encoded by an amino acid sequence as shown in SEQ ID NO:2.

36. An isolated pancreatic T-type calcium channel protein encoded by a first amino acid sequence having at least 90% amino acid identity to a second amino acid sequence, said second amino acid sequence as shown in SEQ ID NO:2.

37. An antibody or fragment thereof specific for the pancreatic T-type calcium channel protein of claim 36.

38. The antibody of claim 37 wherein said antibody comprises a monoclonal antibody.

39. The antibody of claim 37 wherein said antibody comprises a polyclonal antibody.

40. A composition comprising the pancreatic T-type calcium channel protein of claim 36 and a compatible carrier.

41. A method of detecting presence of a pancreatic T-type calcium channel protein in a sample, said method comprising:
contacting a sample with the antibody or fragment thereof of claim 37, wherein said antibody or fragment thereof binds to any of said pancreatic T-type calcium channel protein present in said sample, forming a complex therewith; and
detecting said complex, thereby detecting presence of a pancreatic T-type calcium channel protein in said sample.

42. The method of claim 41 wherein said antibody or fragment thereof is labeled with a detectable marker.

43. A method of modifying insulin secretion by pancreatic beta cells, the method comprising modifying levels of functional T type calcium channels in the pancreatic beta cells.

44. The method of claim 43 wherein modifying levels of functional T type calcium channels comprises modifying T type calcium channel gene expression in the pancreatic beta cells.

45. The method of claim 44 wherein modifying T type calcium channel gene expression comprises exposing the pancreatic beta cells to a compound which modifies T type calcium channel gene expression.

46. The method of claim 45 wherein the compound is an antisense oligonucleotide targeted to the T type calcium channel gene.

47. The method of claim 43 wherein modifying levels of functional T type calcium channel comprises exposing the pancreatic beta cells to an inhibitor of the functional T type calcium channel.

48. The method of claim 43 wherein modifying levels of functional T type calcium channel comprises exposing the pancreatic beta cells to a compound which interferes with membrane T type calcium channel formation.

49. The method of claim 43 wherein the pancreatic beta cells are present in a subject having type II diabetes.

50. A method of treating type II diabetes in a subject, the method comprising administering to the subject an amount of a compound effective to modify levels of functional T type calcium channel in the pancreatic beta cells of the subject.

51. The method of claim 50 wherein the compound modifies levels of functional T type calcium channel by modifying T type calcium channel gene expression.

52. The method of claim 51 wherein modifying T type calcium channel gene expression comprises exposing the pancreatic beta cells to a compound which modifies T type calcium channel gene expression.

53. The method of claim 52 wherein the compound is an antisense oligonucleotide targeted to the T type calcium channel gene.

54. The method of claim 50 wherein the compound is an inhibitor of the functional T type calcium channel.

55. The method of claim 50 wherein the compound interferes with membrane T type calcium channel formation.

56. A method of modifying basal calcium levels in cells, the method comprising modifying levels of functional T type calcium channels in the cells.

57. A method of modifying the action potential of L type calcium channels in cells, the method comprising modifying levels of functional T type calcium channels in the cells.

58. A method of modifying pancreatic beta cell death, the method comprising modifying levels of functional T type calcium channels in the pancreatic beta cells.

59. A method of modifying pancreatic beta cell proliferation, the method comprising modifying levels of functional T type calcium channels in the pancreatic beta cells.

60. A method of modifying calcium influx through L type calcium channels in cells, the method comprising modifying levels of functional T type calcium channels in the cells.


SEQUENCE LISTING (11 sequences)

SEQ ID NO: 1 - DNA, 7129 bp, Rattus sp.
atggacgaggaggaggatggagcgggcgccgaggagtcgggacagccccgtagcttcacgcagctcaacgacctgtccggggccgggggccggcaggggccggggtcgacggaaaaggacccgggcagcgcggactccgaggcggaggggctgccgtacccggcgctagccccggtggttttcttctacttgagccaggacagccgcccgcggagctggtgtctccgcacggtctgtaacccgtggttcgagcgagtcagtatgctggtcattcttctcaactgtgtgactctgggtatgttcaggccgtgtgaggacattgcctgtgactcccagcgctgccggatcctgcaggccttcgatgacttcatctttgccttctttgctgtggaaatggtggtgaagatggtggccttgggcatctttgggaagaaatgttacctgggagacacttggaaccggcttgactttttcattgtcattgcagggatgctggagtattcgctggacctgcagaacgtcagcttctccgcagtcaggacagtccgtgtgctgcgaccgctcagggccattaaccgggtgcccagcatgcgcattctcgtcacattactgctggacaccttgcctatgctgggcaacgtcctgctgctctgtttcttcgtctttttcatctttggcatcgtgggcgtccagctgtgggcaggactgcttcgcaaccgatgcttcctccccgagaacttcagcctccccctgagcgtggacctggagccttattaccagacagagaatgaggacgagagccccttcatctgctctcagcctcgggagaatggcatgagatcctgcaggagtgtgcccacactgcgtggggaaggcggtggtggcccaccctgcagtctggactatgagacctataacagttccagcaacaccacctgtgtcaactggaaccagtactataccaactgctctgcgggcgagcacaaccccttcaaaggcgccatcaactttgacaacattggctatgcctggatcgccatcttccaggtcatcacactggagggctgggtcgacatcatgtacttcgtaatggacgctcactccttctacaacttcatctacttcattcttctcatcatcgtgggctccttcttcatgatcaacctgtgcctggtggtgattgccacgcagttctccgagaccaaacagcgggagagtcagctgatgcgggagcagcgtgtacgattcctgtccaatgctagcaccctggcaagcttctctgagccaggcagctgctatgaggagctactcaagtacctggtgtacatcctccgaaaagcagcccgaaggctggcccaggtctctagggctataggcgtgcgggctgggctgctcagcagcccagtggcccgtagtgggcaggagccccagcccagtggcagctgcactcgctcacaccgtcgtctgtctgtccaccacctggtccaccaccatcaccaccaccatcaccactaccacctgggtaatgggacgctcagagttccccgggccagcccagagatccaggacagggatgccaatgggtctcgccggctcatgctaccaccaccctctacacccactccctctgggggccctccgaggggtgcggagtctgtacacagcttctaccatgctgactgccacttggagccagtccgttgccaggcaccccctcccagatgcccatcggaggcatctggtaggactgtgggtagtgggaaggtgtaccccactgtgcataccagccctccaccagagatactgaaggataaagcactagtggaggtggcccccagccctgggccccccaccctcaccagcttcaacatcccacctgggcccttcagctccatgcacaagctcctggagacacagagtacgggagcctgccatagctcctgcaaaatctccagcccttgctccaaggcagacagtggagcctgcgggccggacagttgtccctactgtgcccggacaggagcaggagagccagagtccgctgaccatgtcatgcctgactcagacagcgaggctgtgtatgagttcacacaggacgctcagcacagtgacctccgggatccccacagccggcggcgacagcggagcctgggcccagatgcagagcctagttctgtgctggctttctggaggctgatctgtgacacattccggaagatcgtagatagcaaatactttggccggggaatcatgatcgccatcctggtcaatacactcagcatgggcatcgagtaccacgagcagcccgaggagctcaccaacgccctggaaatcagcaacatcgtcttcaccagcctcttcgccttggagatgctgctgaaactgcttgtctacggtccctttggctacattaagaatccctacaacatctttgatggtgtcattgtggtcatcagtgtgtgggagattgtgggccagcagggaggtggcctgtcggtgctgcggaccttccgcctgatgcgggtgctgaagctggtgcgcttcctgccggccctgcagcgccagctcgtggtgctcatgaagaccatggacaacgtggccaccttctgcatgctcctcatgctgttcatcttcatcttcagcatcctgggcatgcatctctttggttgcaagttcgcatctgaacgggatggggacacgttgccagaccggaagaatttcgactccctgctctgggccatcgtcactgtctttcagattctgactcaggaagactggaataaagtcctctacaacggcatggcctccacatcgtcttgggctgctctttacttcatcgccctcatgacttttggcaactatgtgctctttaacctgctggtggccattcttgtggaaggattccaggcagaggaaatcggcaaacgggaagatgcgagtggacagttaagctgtattcagctgcctgtcaactctcaggggggagatgccaccaagtctgagtcagagcctgatttcttttcgcccagtgtggatggtgatggggacagaaagaagcgcttggccctggtggctttgggagaacacgcggaactacgaaagagccttttgccacccctcatcatccatacggctgcgacaccaatgtcactacccaagagctccagcacaggtgtgggggaagcactgggctctggctctcgacgtaccagtagcagtgggtccgctgagcctggagctgcccaccatgagatgaaatctccgccaagtgcccgcagctccccgcacagtccctggagtgcggcaagcagctggaccagcaggcgctccagcaggaacagcctgggccgggcccccagcctaaagcggaggagcccgagcggggagcggaggtccctgctgtctggagagggccaggagagtcaggatgaggaggaaagttcagaagaggaccgggccagcccagcaggcagtgaccatcgccacaggggttccttggaacgtgaggccaagagttcctttgacctgcctgacactctgcaggtgccggggctgcaccgcacagccagcggccggagctctgcctctgagcaccaagactgtaatggcaagtcggcttcagggcgtttggcccgcaccctgaggactgatgacccccaactggatggggatgatgacaatgatgagggaaatctgagcaaaggggaacgcatacaagcctgggtcagatcccggcttcctgcctgttgccgagagcgagattcctggtcggcctatatctttcctcctcagtcaaggtttcgtctcctgtgtcaccggatcatcacccacaagatgtttgaccatgtggtcctcgtcatcatcttcctcaactgtatcaccatcgctatggagcgccccaaaattgacccccacagcgctgagcgcatcttcctgaccctctccaactacatcttcacggcagtctttctagctgaaatgacagtgaaggtggtggcactgggctggtgctttggggagcaggcctacctgcgcagcagctggaatgtgctggacggcttgctggtgctcatctccgtcatcgacatcctggtctccatggtctccgacagcggcaccaagatccttggcatgctgagggtgctgcggctgctgcggaccctgcgtccactcagggtcatcagccgggcccagggactgaagctggtggtagagactctgatgtcatccctcaaacccattggcaacattgtggtcatttgctgtgccttcttcatcatttttggaattctcggggtgcagctcttcaaagggaagttcttcgtgtgtcagggtgaggacaccaggaacatcactaacaaatccgactgcgctgaggccagctaccgatgggtccggcacaagtacaactttgacaacctgggccaggctctgatgtccctgtttgtgctggcctccaaggatggttgggttgacatcatgtatgatgggctggatgctgtgggtgtggatcagcagcccatcatgaaccacaacccctggatgctgctatacttcatctccttcctcctcatcgtggccttctttgtcctgaacatgtttgtgggcgtggtggtggagaacttccataagtgcagacagcaccaggaggaggaggaggcgaggcggcgtgaggagaagcgactacggaggctggagaaaaagagaaggaatctaatgttggacgatgtaattgcttccggcagctcagccagcgctgcgtcagaagcccagtgcaagccctactactctgactactcgagattccggctccttgtccaccacctgtgtaccagccactacctggacctcttcatcactggtgtcatcgggctgaacgtggtcactatggccatggaacattaccagcagccccagatcctggacgaggctctgaagatctgcaattacatctttaccgtcatctttgtctttgagtcagttttcaaacttgtggcctttggcttccgccgtttcttccaggacaggtggaaccagctggacctggctattgtgcttctgtccatcatgggcatcacactggaggagattgaggtcaatgcttcgctgcccatcaaccccaccatcatccgtatcatgagggtgctccgcattgctcgagttctgaagctgttgaagatggctgtgggcatgcgggcactgctggacacggtgatgcaggccctgccccaggtggggaacctgggacttctcttcatgttattgtttttcatctttgcagctctgggcgtggagctctttggagacctggagtgtgatgagacacacccttgtgagggcttgggtcggcatgccacctttaggaactttggtatggcctttctgaccctcttccgagtctccactggtgacaactggaatggtattatgaaggacaccctccgggactgtgaccaggagtccacctgctacaacactgtcatctcccctatctactttgtgtccttcgtgctgacggcccagtttgtgctggtcaacgtggtcatagctgtgctgatgaagcacctggaagaaagcaacaaagaggccaaggaggaggccgagctcgaggccgagctggagctggagatgaagacgctcagcccgcagccccactccccgctgggcagccccttcctctggcccggggtggagggtgtcaacagtcctgacagccctaagcctggggctccacacaccactgcccacattggagcagcctcgggcttctcccttgagcaccccacgatggtaccccaccccgaggaggtgccagtccccctaggaccagacctgctgactgtgaggaagtctggtgtcagccggacgcactctctgcccaatgacagctacatgtgccgcaatgggagcactgctgagagatccctaggacacaggggctgggggctccccaaagcccagtcaggctccatcttgtccgttcactcccaaccagcagacaccagctgcatcctacagcttcccaaagatgtgcactatctgctccagcctcatggggcccccacctggggcgccatccctaaactacccccacctggccgctcccctctggctcagaggcctctcaggcgccaggcagcaataaggactgactccctggatgtgcagggcctgggtagccgggaagacctgttgtcagaggtgagtgggccctcctgccctctgacccggtcctcatccttctggggcgggtcgagcatccaggtgcagcagcgttccggcatccagagcaaagtctccaagcacatccgcctgccagccccttgcccaggcctggaacccagctgggccaaggaccctccagagaccagaagcagcttagagctggacacggagctgagctggatttcaggagacctccttcccagcagccaggaagaacccctgtccccacgggacctgaagaagtgctacagtgtagagacccagagctgcaggcgcaggcctgggtcctggctagatgaacagcggagacactccattgctgtcagctgtctggacagcggctcccaaccccgcctatgtccaagcccctcaagcctcgggggccaacctcttgggggtcctgggagccggcctaagaaaaaactcagcccacccagtatctctatagaccccccggagagccagggctctcggcccccatgcagtcctggtgtctgcctcaggaggagggcgccggccagtgactctaaggatccctcggtctccagcccccttgacagcacggctgcctcaccctccccaaagaaagacacgctgagtctctctggtttgtcttctgacccaacagacatggacccctgagtcctacccactctcccccatcacctttctccaccgggtgcagatcctagctccgcctcctgggcagcgtttctgaaaagtcccacgtaagcagcaagcagccacgaggcacctcacctgccttcttcagtggctggtggggatgacgagcagaacttccggagagtcgatctgaagagaacacagccctggagcccctgcctccgggaagaaggaaaaggagaaagcccagtgtggccaaggctcccgacaccaggagctg

SEQ ID NO: 2 - protein, 2374 aa, Rattus sp.
MDEEEDGAGAEESGQPRSFTQLNDLSGAGGRQGPGSTEKDPGSADSEAEGLPYPALAPVVFFYLSQDSRPRSWCLRTVCNPWFERVSMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLEPYYQTENEDESPFICSQPRENGMRSCRSVPTLRGEGGGGPPCSLDYETYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAIGVRAGLLSSPVARSGQEPQPSGSCTRSHRRLSVHHLVHHHHHHHHHYHLGNGTLRVPRASPEIQDRDANGSRRLMLPPPSTPTPSGGPPRGAESVHSFYHADCHLEPVRCQAPPPRCPSEASGRTVGSGKVYPTVHTSPPPEILKDKALVEVAPSPGPPTLTSFNIPPGPFSSMHKLLETQSTGACHSSCKISSPCSKADSGACGPDSCPYCARTGAGEPESADHVMPDSDSEAVYEFTQDAQHSDLRDPHSRRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEEIGKREDASGQLSCIQLPVNSQGGDATKSESEPDFFSPSVDGDGDRKKRLALVALGEHAELRKSLLPPLIIHTAATPMSLPKSSSTGVGEALGSGSRRTSSSGSAEPGAAHHEMKSPPSARSSPHSPWSAASSWTSRRSSRNSLGRAPSLKRRSPSGERRSLLSGEGQESQDEEESSEEDRASPAGSDHRHRGSLEREAKSSFDLPDTLQVPGLHRTASGRSSASEHQDCNGKSASGRLARTLRTDDPQLDGDDDNDEGNLSKGERIQAWVRSRLPACCRERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRNLMLDDVIASGSSASAASEAQCKPYYSDYSRFRLLVHHLCTSHYLDLFITGVIGLNVVTMAMEHYQQPQILDEALKICNYIFTVIFVFESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGVNSPDSPKPGAPHTTAHIGAASGFSLEHPTMVPHPEEVPVPLGPDLLTVRKSGVSRTHSLPNDSYMCRNGSTAERSLGHRGWGLPKAQSGSILSVHSQPADTSCILQLPKDVHYLLQPHGAPTWGAIPKLPPPGRSPLAQRPLRRQAAIRTDSLDVQGLGSREDLLSEVSGPSCPLTRSSSFWGGSSIQVQQRSGIQSKVSKHIRLPAPCPGLEPSWAKDPPETRSSLELDTELSWISGDLLPSSQEEPLSPRDLKKCYSVETQSCRRRPGSWLDEQRRHSIAVSCLDSGSQPRLCPSPSSLGGQPLGGPGSRPKKKLSPPSISIDPPESQGSRPPCSPGVCLRRRAPASDSKDPSVSSPLDSTAASPSPKKDTLSLSGLSSDPTDMDPVLPTLPHHLSPPGADPSSASWAAFLKSPTAASSHEAPHLPSSVAGGDDEQNFRRVDLKRTQPWSPCLREEGKGESPVWPRLPTPGA

SEQ ID NO: 3 - DNA, 7285 bp, Rattus sp.
gagctgagctgaactggccctcctggggactcagcaagctctctagagccccccacatgctcccccaccggggtcccccggttgcgtgaggacacctcctctgaggggctccgctcgcccctcttcggaccccccggggccccggctggccagaggatggacgaggaggaggatggagcgggcgccgaggagtcgggacagccccgtagcttcacgcagctcaacgacctgtccggggccgggggccggcaggggccggggtcgacggaaaaggacccgggcagcgcggactccgaggcggaggggctgccgtacccggcgctagccccggtggttttcttctacttgagccaggacagccgcccgcggagctggtgtctccgcacggtctgtaacccgtggttcgagcgagtcagtatgctggtcattcttctcaactgtgtgactctgggtatgttcaggccgtgtgaggacattgcctgtgactcccagcgctgccggatcctgcaggccttcgatgacttcatctttgccttctttgctgtggaaatggtggtgaagatggtggccttgggcatctttgggaagaaatgttacctgggagacacttggaaccggcttgactttttcattgtcattgcagggatgctggagtattcgctggacctgcagaacgtcagcttctccgcagtcaggacagtccgtgtgctgcgaccgctcagggccattaaccgggtgcccagcatgcgcattctcgtcacattactgctggacaccttgcctatgctgggcaacgtcctgctgctctgtttcttcgtctttttcatctttggcatcgtgggcgtccagctgtgggcaggactgcttcgcaaccgatgcttcctccccgagaacttcagcctccccctgagcgtggacctggagccttattaccagacagagaatgaggacgagagccccttcatctgctctcagcctcgggagaatggcatgagatcctgcaggagtgtgcccacactgcgtggggaaggcggtggtggcccaccctgcagtctggactatgagacctataacagttccagcaacaccacctgtgtcaactggaaccagtactataccaactgctctgcgggcgagcacaaccccttcaaaggcgccatcaactttgacaacattggctatgcctggatcgccatcttccaggtcatcacactggagggctgggtcgacatcatgtacttcgtaatggacgctcactccttctacaacttcatctacttcattcttctcatcatcgtgggctccttcttcatgatcaacctgtgcctggtggtgattgccacgcagttctccgagaccaaacagcgggagagtcagctgatgcgggagcagcgtgtacgattcctgtccaatgctagcaccctggcaagcttctctgagccaggcagctgctatgaggagctactcaagtacctggtgtacatcctccgaaaagcagcccgaaggctggcccaggtctctagggctataggcgtgcgggctgggctgctcagcagcccagtggcccgtagtgggcaggagccccagcccagtggcagctgcactcgctcacaccgtcgtctgtctgtccaccacctggtccaccaccatcaccaccaccatcaccactaccacctgggtaatgggacgctcagagttccccgggccagcccagagatccaggacagggatgccaatgggtctcgccggctcatgctaccaccaccctctacacccactccctctgggggccctccgaggggtgcggagtctgtacacagcttctaccatgctgactgccacttggagccagtccgttgccaggcaccccctcccagatgcccatcggaggcatctggtaggactgtgggtagtgggaaggtgtaccccactgtgcataccagccctccaccagagatactgaaggataaagcactagtggaggtggcccccagccctgggccccccaccctcaccagcttcaacatcccacctgggcccttcagctccatgcacaagctcctggagacacagagtacgggagcctgccatagctcctgcaaaatctccagcccttgctccaaggcagacagtggagcctgcgggccggacagttgtccctactgtgcccggacaggagcaggagagccagagtccgctgaccatgtcatgcctgactcagacagcgaggctgtgtatgagttcacacaggacgctcagcacagtgacctccgggatccccacagccggcggcgacagcggagcctgggcccagatgcagagcctagttctgtgctggctttctggaggctgatctgtgacacattccggaagatcgtagatagcaaatactttggccggggaatcatgatcgccatcctggtcaatacactcagcatgggcatcgagtaccacgagcagcccgaggagctcaccaacgccctggaaatcagcaacatcgtcttcaccagcctcttcgccttggagatgctgctgaaactgcttgtctacggtccctttggctacattaagaatccctacaacatctttgatggtgtcattgtggtcatcagtgtgtgggagattgtgggccagcagggaggtggcctgtcggtgctgcggaccttccgcctgatgcgggtgctgaagctggtgcgcttcctgccggccctgcagcgccagctcgtggtgctcatgaagaccatggacaacgtggccaccttctgcatgctcctcatgctgttcatcttcatcttcagcatcctgggcatgcatctctttggttgcaagttcgcatctgaacgggatggggacacgttgccagaccggaagaatttcgactccctgctctgggccatcgtcactgtctttcagattctgactcaggaagactggaataaagtcctctacaacggcatggcctccacatcgtcttgggctgctctttacttcatcgccctcatgacttttggcaactatgtgctctttaacctgctggtggccattcttgtggaaggattccaggcagaggaaatcggcaaacgggaagatgcgagtggacagttaagctgtattcagctgcctgtcaactctcaggggggagatgccaccaagtctgagtcagagcctgatttcttttcgcccagtgtggatggtgatggggacagaaagaagcgcttggccctggtggctttgggagaacacgcggaactacgaaagagccttttgccacccctcatcatccatacggctgcgacaccaatgtcactacccaagagctccagcacaggtgtgggggaagcactgggctctggctctcgacgtaccagtagcagtgggtccgctgagcctggagctgcccaccatgagatgaaatctccgccaagtgcccgcagctccccgcacagtccctggagtgcggcaagcagctggaccagcaggcgctccagcaggaacagcctgggccgggcccccagcctaaagcggaggagcccgagcggggagcggaggtccctgctgtctggagagggccaggagagtcaggatgaggaggaaagttcagaagaggaccgggccagcccagcaggcagtgaccatcgccacaggggttccttggaacgtgaggccaagagttcctttgacctgcctgacactctgcaggtgccggggctgcaccgcacagccagcggccggagctctgcctctgagcaccaagactgtaatggcaagtcggcttcagggcgtttggcccgcaccctgaggactgatgacccccaactggatggggatgatgacaatgatgagggaaatctgagcaaaggggaacgcatacaagcctgggtcagatcccggcttcctgcctgttgccgagagcgagattcctggtcggcctatatctttcctcctcagtcaaggtttcgtctcctgtgtcaccggatcatcacccacaagatgtttgaccatgtggtcctcgtcatcatcttcctcaactgtatcaccatcgctatggagcgccccaaaattgacccccacagcgctgagcgcatcttcctgaccctctccaactacatcttcacggcagtctttctagctgaaatgacagtgaaggtggtggcactgggctggtgctttggggagcaggcctacctgcgcagcagctggaatgtgctggacggcttgctggtgctcatctccgtcatcgacatcctggtctccatggtctccgacagcggcaccaagatccttggcatgctgagggtgctgcggctgctgcggaccctgcgtccactcagggtcatcagccgggcccagggactgaagctggtggtagagactctgatgtcatccctcaaacccattggcaacattgtggtcatttgctgtgccttcttcatcatttttggaattctcggggtgcagctcttcaaagggaagttcttcgtgtgtcagggtgaggacaccaggaacatcactaacaaatccgactgcgctgaggccagctaccgatgggtccggcacaagtacaactttgacaacctgggccaggctctgatgtccctgtttgtgctggcctccaaggatggttgggttgacatcatgtatgatgggctggatgctgtgggtgtggatcagcagcccatcatgaaccacaacccctggatgctgctatacttcatctccttcctcctcatcgtggccttctttgtcctgaacatgtttgtgggcgtggtggtggagaacttccataagtgcagacagcaccaggaggaggaggaggcgaggcggcgtgaggagaagcgactacggaggctggagaaaaagagaaggaatctaatgttggacgatgtaattgcttccggcagctcagccagcgctgcgtcagaagcccagtgcaagccctactactctgactactcgagattccggctccttgtccaccacctgtgtaccagccactacctggacctcttcatcactggtgtcatcgggctgaacgtggtcactatggccatggaacattaccagcagccccagatcctggacgaggctctgaagatctgcaattacatctttaccgtcatctttgtctttgagtcagttttcaaacttgtggcctttggcttccgccgtttcttccaggacaggtggaaccagctggacctggctattgtgcttctgtccatcatgggcatcacactggaggagattgaggtcaatgcttcgctgcccatcaaccccaccatcatccgtatcatgagggtgctccgcattgctcgagttctgaagctgttgaagatggctgtgggcatgcgggcactgctggacacggtgatgcaggccctgccccaggtggggaacctgggacttctcttcatgttattgtttttcatctttgcagctctgggcgtggagctctttggagacctggagtgtgatgagacacacccttgtgagggcttgggtcggcatgccacctttaggaactttggtatggcctttctgaccctcttccgagtctccactggtgacaactggaatggtattatgaaggacaccctccgggactgtgaccaggagtccacctgctacaacactgtcatctcccctatctactttgtgtccttcgtgctgacggcccagtttgtgctggtcaacgtggtcatagctgtgctgatgaagcacctggaagaaagcaacaaagaggccaaggaggaggccgagctcgaggccgagctggagctggagatgaagacgctcagcccgcagccccactccccgctgggcagccccttcctctggcccggggtggagggtgtcaacagtcctgacagccctaagcctggggctccacacaccactgcccacattggagcagcctcgggcttctcccttgagcaccccacgatggtaccccaccccgaggaggtgccagtccccctaggaccagacctgctgactgtgaggaagtctggtgtcagccggacgcactctctgcccaatgacagctacatgtgccgcaatgggagcactgctgagagatccctaggacacaggggctgggggctccccaaagcccagtcaggctccatcttgtccgttcactcccaaccagcagacaccagctgcatcctacagcttcccaaagatgtgcactatctgctccagcctcatggggcccccacctggggcgccatccctaaactacccccacctggccgctcccctctggctcagaggcctctcaggcgccaggcagcaataaggactgactccctggatgtgcagggcctgggtagccgggaagacctgttgtcagaggtgagtgggccctcctgccctctgacccggtcctcatccttctggggcgggtcgagcatccaggtgcagcagcgttccggcatccagagcaaagtctccaagcacatccgcctgccagccccttgcccaggcctggaacccagctgggccaaggaccctccagagaccagaagcagcttagagctggacacggagctgagctggatttcaggagacctccttcccagcagccaggaagaacccctgtccccacgggacctgaagaagtgctacagtgtagagacccagagctgcaggcgcaggcctgggtcctggctagatgaacagcggagacactccattgctgtcagctgtctggacagcggctcccaaccccgcctatgtccaagcccctcaagcctcgggggccaacctcttgggggtcctgggagccggcctaagaaaaaactcagcccacccagtatctctatagaccccccggagagccagggctctcggcccccatgcagtcctggtgtctgcctcaggaggagggcgccggccagtgactctaaggatccctcggtctccagcccccttgacagcacggctgcctcaccctccccaaagaaagacacgctgagtctctctggtttgtcttctgacccaacagacatggacccctgagtcctacccactctcccccatcacctttctccaccgggtgcagatcctagctccgcctcctgggcagcgtttctgaaaagtcccacgtaagcagcaagcagccacgaggcacctcacctgccttcttcagtggctggtggggatgacgagcagaacttccggagagtcgatctgaagagaacacagccctggagcccctgcctccgggaagaaggaaaaggagaaagcccagtgtggccaaggctcccgacaccaggagctg

SEQ ID NO: 4 - protein, 2425 aa, Rattus sp.
ELSTGPPGDSASSLEPPTCSPTGVPRLREDTSSEGLRSPLFGPPGAPAGQRMDEEEDGAGAEESGQPRSFTQLNDLSGAGGRQGPGSTEKDPGSADSEAEGLPYPALAPVVFFYLSQDSRPRSWCLRTVCNPWFERVSMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLEPYYQTENEDESPFICSQPRENGMRSCRSVPTLRGEGGGGPPCSLDYETYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAIGVRAGLLSSPVARSGQEPQPSGSCTRSHRRLSVHHLVHHHHHHHHHYHLGNGTLRVPRASPEIQDRDANGSRRLMLPPPSTPTPSGGPPRGAESVHSFYHADCHLEPVRCQAPPPRCPSEASGRTVGSGKVYPTVHTSPPPEILKDKALVEVAPSPGPPTLTSFNIPPGPFSSMHKLLETQSTGACHSSCKISSPCSKADSGACGPDSCPYCARTGAGEPESADHVMPDSDSEAVYEFTQDAQHSDLRDPHSRRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEEIGKREDASGQLSCIQLPVNSQGGDATKSESEPDFFSPSVDGDGDRKKRLALVALGEHAELRKSLLPPLIIHTAATPMSLPKSSSTGVGEALGSGSRRTSSSGSAEPGAAHHEMKSPPSARSSPHSPWSAASSWTSRRSSRNSLGRAPSLKRRSPSGERRSLLSGEGQESQDEEESSEEDRASPAGSDHRHRGSLEREAKSSFDLPDTLQVPGLHRTASGRSSASEHQDCNGKSASGRLARTLRTDDPQLDGDDDNDEGNLSKGERIQAWVRSRLPACCRERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRNLMLDDVIASGSSASAASEAQCKPYYSDYSRFRLLVHHLCTSHYLDLFITGVIGLNVVTMAMEHYQQPQILDEALKICNYIFTVIFVFESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGVNSPDSPKPGAPHTTAHIGAASGFSLEHPTMVPHPEEVPVPLGPDLLTVRKSGVSRTHSLPNDSYMCRNGSTAERSLGHRGWGLPKAQSGSILSVHSQPADTSCILQLPKDVHYLLQPHGAPTWGAIPKLPPPGRSPLAQRPLRRQAAIRTDSLDVQGLGSREDLLSEVSGPSCPLTRSSSFWGGSSIQVQQRSGIQSKVSKHIRLPAPCPGLEPSWAKDPPETRSSLELDTELSWISGDLLPSSQEEPLSPRDLKKCYSVETQSCRRRPGSWLDEQRRHSIAVSCLDSGSQPRLCPSPSSLGGQPLGGPGSRPKKKLSPPSISIDPPESQGSRPPCSPGVCLRRRAPASDSKDPSVSSPLDSTAASPSPKKDTLSLSGLSSDPTDMDPVLPTLPHHLSPPGADPSSASWAAFLKSPTAASSHEAPHLPSSVAGGDDEQNFRRVDLKRTQPWSPCLREEGKGESPVWPRLPTPGA

SEQ ID NO: 5 - protein, 7 aa, Rattus sp.
SKEKQMA

SEQ ID NO: 6 - DNA, 17 bp, Artificial Sequence
  Description of Artificial Sequence Primer Sequence
tngchatggagmgnccy

SEQ ID NO: 7 - DNA, 18 bp, Artificial Sequence
  Description of Artificial Sequence Primer Sequence
cttbcccttgaasarctg

SEQ ID NO: 8 - DNA, 24 bp, Artificial Sequence
  Description of Artificial Sequence Primer Sequence
ccgctgtcggagaccatggagacc

SEQ ID NO: 9 - DNA, 25 bp, Artificial Sequence
  Description of Artificial Sequence Primer Sequence
agcggcccaaaattgacccccacag

SEQ ID NO: 10 - DNA, 18 bp, Artificial Sequence
  Description of Artificial Sequence Primer Sequence
gaagatgcgagtggacag

SEQ ID NO: 11 - DNA, 18 bp, Artificial Sequence
  Description of Artificial Sequence Primer Sequence
ctgtggcgatggtcactg